(19) United States
(12) Patent Application Publication
(10) Pub. No.: US 2012/0308544 A1
(43) Pub. Date: Dec. 6, 2012

(54) Substances and Methods for the Treatment of Lysosmal Storage Diseases

(76) Inventor: Robert Steinfeld, Gottingen (DE)

(21) Appl. No.: 13/515,355

(22) PCT Filed: Dec. 14, 2010
(86) PCT No.: PCT/EP10/69649

§ 371 Date: Aug. 23, 2012

(30) Foreign Application Priority Data
Dec. 14, 2009 (EP) 09179047.7

(51) Int. Cl. A61K 38/48; A61P 3/00; C12N 9/96; C12N 15/62

(52) U.S. Cl. 424/94.3; 536/23.2; 435/188

(57) ABSTRACT

The present invention relates to a chimeric molecule comprising (i) a targeting moiety that binds to heparin or heparan sulfate proteoglycans, (ii) a lysosomal peptide or protein, (iii) wherein the targeting moiety is a neurotrophic growth factor and/or, wherein the targeting moiety comprises one of the following consensus sequences BBXB, BXBB, BBXXB, BXXBB, BBXXXB or BXXXBB and wherein B represents an arginine, lysine or histidine amino acid and X represents any amino acid, (iii) with the proviso that the targeting moiety is at least thirteen amino acids long.

### FIELD OF THE INVENTION

This invention is in the field of biology and medicine in particular human therapeutics, more in particular in the field of lysosomal storage diseases (LSDs) which are a group of approximately 40 rare inherited metabolic disorders that result from defects in lysosomal function. Lysosomal storage diseases result when a specific component of lysosomes which are organelles in the body's cells malfunctions.

### BACKGROUND OF THE INVENTION

Lysosomal storage diseases (LSDs) are a group of approximately 40 rare inherited metabolic disorders that result from defects in lysosomal function.

Tay-Sachs disease was the first of these disorders to be described, in 1881, followed by Gaucher disease in 1882 and Fabry disease in 1898. In the late 1950s and early 1960s, de Duve and colleagues, using cell fractionation techniques, cytological studies and biochemical analyses, identified and characterized the lysosome as a cellular organelle responsible for intracellular digestion and recycling of macromolecules. Pompe disease was the first disease to be identified as a LSD in 1963 (α-glucosidase deficiency).

Lysosomal storage disorders are caused by lysosomal dysfunction usually as a consequence of deficiency of a single enzyme required for the metabolism of lipids, proteins or carbohydrates. Worldwide, individual LSDs occur with incidences of less than 1:100.000, however, as a group the incidence is about 1:5000-1:10.000. Lysosomal disorders are caused by partial or complete loss of function of lysosomal proteins, mostly lysosomal enzymes. When this happens, substances accumulate in the cell. In other words, when the lysosome doesn't function normally, excess products destined for breakdown and recycling are stored in the cell.

Lysosomal storage diseases affect mostly children and they often die at a young and unpredictable age, many within a few months or years of birth. Many other children die of this disease following years of suffering from various symptoms of their particular disorder. The symptoms of lysosomal storage disease vary, depending on the particular disorder and other environmental and genetic factors. Usually, early onset forms are associated with a severe phenotype whereas late onset forms show a milder phenotype. Typical symptoms can include developmental delay, movement disorders, seizures, dementia, deafness and/or blindness. Some people with lysosomal storage disease have enlarged livers (hepatomegaly) and enlarged spleens (splenomegaly), pulmonary and cardiac problems, and bones that grow abnormally.

The lysosomal storage diseases are generally classified by the nature of the primary stored material involved, and can be broadly broken into the following: (ICD-10 codes are provided), (I) (E75) lipid storage disorders, mainly sphingolipidoses (including Gaucher's and Niemann-Pick diseases), (ii) (E75.0-E75.1) gangliosidosis (including Tay-Sachs disease), (iii) (E75.2) leukodystrophies, (iv) (E76.0) mucopolysaccharidoses (including Hunter syndrome and Hurler disease), (v) (E77) glycoprotein storage disorders and (vi) (E77.0-E77.1) mucolipidoses.

Alternatively to the protein targets, lysosomal storage diseases may be classified by the type of protein that is deficient and is causing build-up.

| Type of defect | | |
|---|---|---|
| protein | Disease examples | Deficient protein |
| lysosomal | Sphingolipidoses (e.g., | Various |
| hydrolases | gangliosidoses, like | |
| primarily | GM1- and GM2- | |
| | gangliosidoses, Gaucher's | |
| | disease, Fabry disease, | |
| | Niemann-Pick disease, like | |
| | Niemann-Pick disease | |
| | type A and B) | |
| Posttranslational | Multiple sulfatase deficiency | Multiple sulfatases |
| modification of | | |
| enzymes | | |
| Membrane | Mucolipidosis type II and IIIA | N-acetylglucosamine- |
| transport | | 1-phosphate |
| proteins | | transferase |
| Enzyme | Galactosialidosis | Cathepsin A |
| protecting | | |
| proteins | | |
| Soluble | GM2-AP deficiency, | GM2-AP |
| nonenzymatic | variant AB | |
| proteins | | |
| Transmembrane | SAP deficiency | Sphingolipid activator |
| proteins | | proteins |
| | Niemann-Pick disease, type C | NPC1 and NPC2 |
| | Salla disease | Sialin |

There are no cures for lysosomal storage diseases especially for those with brain involvement and treatment is mostly symptomatic, although bone marrow transplantation and enzyme replacement therapy (ERT) have been tried with some success (Clarke J T, Iwanochko R M (2005) “Enzyme replacement therapy of Fabry disease”. Mol. Neurobiol. 32 (1): 43-50 and Bruni S, Loschi L, Incerti C, Gabrielli O, Coppa G V (2007) “Update on treatment of lysosomal storage diseases”, Acta Myol 26 (1): 87-92). In addition, umbilical cord blood transplantation is being performed at specialized centres for a number of these diseases. Further, substrate reduction therapy, a method used to decrease the accumulation of storage material, is currently being evaluated for some of these diseases. Also enzyme replacement therapy is being attempted however; success rates are low because the enzymes are poorly internalized in particular by neurons.

Hence, there is a great need for a therapy for treating individuals that have a lysosomal storage disease, preferably for those lysosomal storage diseases with brain involvement. Such an approach would need to overcome in particular the problem of poor internalization as it was recently shown for example that in rat brain the turnover of mannose-6-phosphate is much lower in the central nervous system than in other tissues.

To improve their internalization and lysosomal targeting beyond the amount mediated by the mannose-6-phosphate pathway the lysosomal proteins have to be modified. This modification can be undertaken by chemical or genetic fusion of the lysosomal protein with other molecules. The resulting chimeric molecules are much better internalized and targeted to the lysosomal compartment than the original, unmodified lysosomal proteins.

The successful fusion of lysosomal enzymes is greatly facilitated by the knowledge of the three-dimensional structure and enzymatic properties of the enzyme. The structures of a couple of lysosomal enzymes have been resolved recently. Among those is tripeptidyl peptidase 1 (TPP1) that has been crystallized as enzymatically active, completely glycosylated full-length protein (s. Pal et al., 2009, structure of tripeptidyl-peptidase I provides insight into the molecular basis of late infantile neuronal ceroid lipofuscinosis. J Biol Chem 284 (6): 3976-84).

### SUMMARY OF THE INVENTION

The inventors of the present invention have astonishingly found that certain chimeric molecules can solve the above mentioned problem. The present invention therefore, relates to a chimeric molecule, comprising (i) a targeting moiety that binds to heparin or heparan sulfate proteoglycans, (ii) a lysosomal peptide or protein and (iii) wherein the targeting moiety is a neurotrophic growth factor and/or, wherein the targeting moiety comprises one of the following consensus sequences BBXB, BXBB, BBXXB, BXXBB, BBXXXB or BXXXBB and wherein B represents an arginine, lysine or histidine amino acid and X represents any amino acid, with the proviso that the targeting moiety is at least thirteen amino acids long.

The invention also relates to a polynucleotide encoding the chimeric molecule according to the invention as well as a pharmaceutical composition comprising a chimeric molecule according to the invention. The chimeric molecule according to the invention is also claimed for the use in the treatment of a disease. In one aspect of the invention the disease is a lysosomal storage disease.

Herein, a “chimeric molecule” is a molecule (preferably a biopolymer) containing molecule portions derived from two different origins, in a preferred embodiment, e.g. from two different genes.

Herein, a “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (e.g. 60%, 70%, 80%, 90%, 95%, 99% or more, calculated using the Smith-Waterman algorithm known by those skilled in the art (Smith & Waterman, 1981). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (e.g. see U.S. Pat. No. 5,753,235).

### FIGURE CAPTIONS

FIG. 1

Purification of the TPP1-FGF2 fusion protein: Coomassie-stained PAGE gel with the 86 kDa TPP1 fusion protein in lane 2 after cation exchange chromatography.

FIG. 2

Autocatalytic processing of the TPP1-FGF2 fusion protein: A Coomassie-stained PAGE gel demonstrating the pH-dependent auto-processing of the 86 kDa TPP1-FGF2 fusion protein after 10 min (10′) and 90 min (90′), respectively. B Activity of the TPP1-FGF2 fusion protein during auto-processing.

FIG. 3

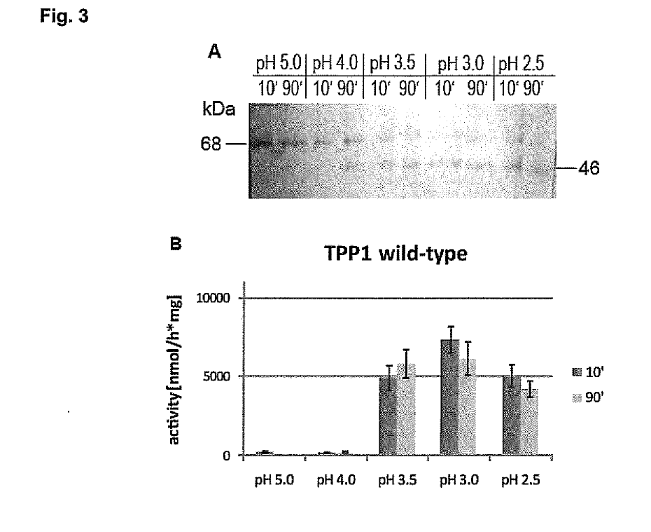

FIG. 3 illustrates the respective auto-processing of the TPP1 wild-type. Interestingly, the TPP1-FGF2 fusion proteins showed a three times higher enzymatic activity than the processed TPP1 wild-type. Since after 10 min of incubation the N-terminal part of TPP1 is preferably cleaved off while the C-terminal part comprising the FGF2 tag is unaffected, it is concluded that the FGF2 tag improves the TPP1 activity. After 90 minutes incubation at room temperature the FGF2 tag is largely cleaved off and the activity is comparable to that of the TPP1 wild-type.

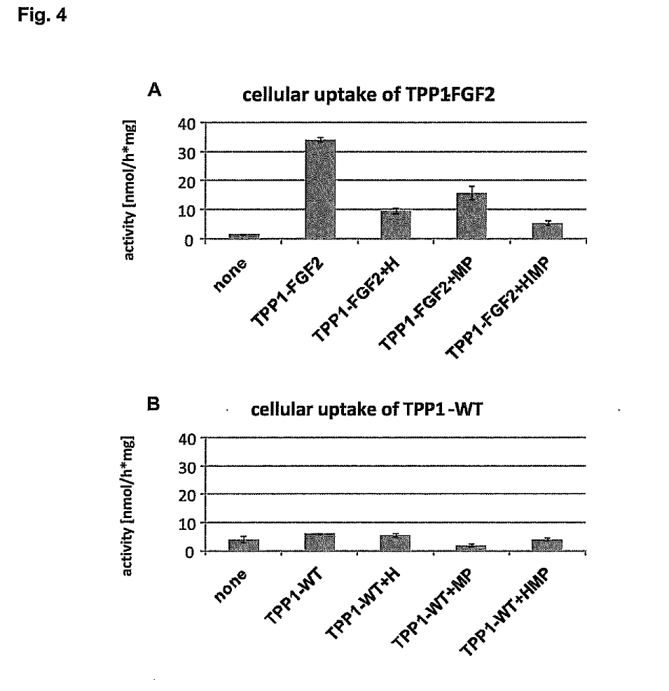

The TPP1-FGF2 fusion protein is significantly more active at pH of 4.0 after a 10 minute (10 times higher) or a 90 minute incubation (5 times higher), respectively, than the TPP1 wild-type. This implies that the FGF2-Tag increases the TPP1 auto-processing at natural lysosomal pH environment (pH 4-5). The in vitro auto-activation at pH 3.5 is not physiological and does not represent the in vivo conditions. In vivo, other interacting compounds such as glycosaminoglycans may increase auto-processing at higher pH (pH 4-5).

FIG. 4

Cellular uptake and intracellular activation of the TPP1-FGF2 fusion protein (A) and the TPP1 wild-type (TPP1-WT) protein (B), respectively. After 48 h of incubation with 0.4 to 0.5 μM TPP1-FGF2 fusion protein or TPP1 wild-type protein, respectively, the activity in the cell lysates of human NT2 cells was determined. TPP1-FGF2 fusion protein treated cells had a six times higher activity than the TPP1 wild-type treated NT2 cells. By adding 1 mM heparin (H) the cellular uptake was reduced to less than 30%, whereas the addition of mannose-6-phosphate (MP) led to a 50% reduction of the uptake of the TPP1-FGF2 fusion protein. The combined addition of H and MP (HMP) led to a reduction to 16% of the cellular uptake/activity of the TPP1-FGF2 fusion protein. For the TPP1 wild-type protein, the highest reduction was observed for MP alone.

FIG. 5

Survival times of tpp1−/− mice under intraventricular injections of either 10 μg TPP1 wild-type or TPP1-FGF2 fusion protein once per week, respectively. Injections were performed from the 30th day of life of the mice.

FIG. 6

FIG. 6 shows testing of the motor coordination of TPP1 wild-type (TPP1-WT) and TPP1-FGF2 fusion protein treated tpp1−/− mice. The time the mice spent on the Rotor Rod before falling down is plotted.

### DETAILED DESCRIPTION OF THE INVENTION

The inventors of the present invention have astonishingly found that certain chimeric molecules can solve the above mentioned problem. The present invention therefore, relates to a chimeric molecule, comprising (i) a targeting moiety that binds to heparin or heparan sulfate proteoglycans, (ii) a lysosomal peptide or protein and (iii) wherein the targeting moiety is a neurotrophic growth factor and/or, wherein the targeting moiety comprises one of the following consensus sequences BBXB, BXBB, BBXXB, BXXBB, BBXXXB or BXXXBB and wherein B represents an arginine, lysine or histidine amino acid and X represents any amino acid, with the proviso that the targeting moiety is at least thirteen amino acids long.

Preferably, the targeting moiety contains at least 7 basic amino acids selected from arginine, lysine and histidine.

It was astonishingly found that chimeric polypeptides according to the invention, such as TPP1-FGF1 fusion proteins showed a significantly higher life expectancy in mice (tpp1−/− mice) as compared to mice treated with the TPP1 wild-type protein.

Moreover, tpp1−/− mice treated with TPP1-FGF2-fusion proteins showed a delayed course of illness in comparison to tpp1−/− mice treated with the TPP1 wild-type. Also motor coordination with the so called Rotor Rod was greatly improved in mice treated with the TPP1-FGF2 fusion protein.

In a preferred embodiment the chimeric molecule of the invention comprises a targeting moiety selected from the group of
(i) annexin II comprising the amino acid sequence according to SEQ ID NO. 1 (KIRSEFKKKYGKSLYY),
(ii) vitronectin comprising the amino acid sequence according to SEQ ID NO. 2 (QRFRHRNRKGYRSQRG),
(iii) ApoB comprising the amino acid sequence according to SEQ ID NO. 3 (KFIIPSPKRPVKLLSG),
(iv) bFGF comprising the amino acid sequence according to SEQ ID NO. 4 (GHFKDPKRLYCKNGGF),
(v) NCAM comprising the amino acid sequence according to SEQ ID NO. 5 (DGGSPIRHYLIKYKAK),
(vi) Protein C inhibitor comprising the amino acid sequence according to SEQ ID NO. 6 (GLSEKTLRKWLKMFKK),
(vii) AT-III comprising the amino acid sequence according to SEQ ID NO. 7 (KLNCRLYRKANKSSKL),
(viii) ApoE comprising the amino acid sequence according to SEQ ID NO. 8 (SHLRKLRKRLLRDADD),
(ix) Fibrin comprising the amino acid sequence according to SEQ ID NO. 9 (GHRPLDKKREEAPSLR),
(x) hGDNF comprising the amino acid sequence according to SEQ ID NO. 10 (SRGKGRRGQRGKNRG),
(xi) B-thromboglobulin comprising the amino acid sequence according to SEQ ID NO. 11 (PDAPRIKKIVQKKLAG)
(xii) Insulin-like growth factor-binding protein-3 comprising the amino acid sequence according to SEQ ID NO. 12 (DKKGFYKKKQCRPSKG),
(xiii) Antp comprising the amino acid sequence according to SEQ ID NO. 13 (RQIKIWFQNRRMKWKK) and
(xiv) human clock comprising the amino acid sequence according to SEQ ID NO. 14 (KRVSRNKSEKKRR).

In one embodiment the growth factor is modified and lysosomal targeting is improved.

In a preferred embodiment the chimeric molecule of the invention is a molecule wherein the targeting moiety and the lysosomal protein or peptide (also referred herein as the enzyme moiety; the two terms may are used interchangeable throughout the whole application) are covalently linked to each other.

Ideally, the chimeric molecule is a single polypeptide chain.

Expression systems for such peptide chains are for example those used with mammalian cells, baculoviruses, and plants.

### Mammalian Systems

Mammalian expression systems are known in the art. A mammalian promoter is any DNA sequence capable of binding mammalian RNA polymerase and initiating the downstream (3′) transcription of a coding sequence (e.g. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation (Sambrook et al. (1989) “Expression of Cloned Genes in Mammalian Cells.” In Molecular Cloning: A Laboratory Manual, 2nd ed.).

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells. The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter (Maniatis et al. (1987) Science 236: 1237; Alberts et al. (1989) Molecular Biology of the Cell, 2nd ed.). Enhancer elements derived from viruses may be particularly useful, because they usually have a broader host range. Examples include the SV40 early gene enhancer (Dijkema et al (1985) EMBO J. 4: 761) and the enhancer/promoters derived from the long terminal repeat (LTR) of the Rous Sarcoma Virus (Gorman et al. (1982b) Proc. Natl. Acad. Sci. 79: 6777) and from human cytomegalovirus (Boshart et al. (1985) Cell 41: 521). Additionally, some enhancers are regulatable and become active only in the presence of an inducer, such as a hormone or metal ion (Sassone-Corsi and Borelli (1986) Trends Genet. 2: 215; Maniatis et al. (1987) Science 236: 1237). A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide. Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells. Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al. (1985) Cell 41: 349; Proudfoot and Whitelaw (1988) “Termination and 3′ end processing of eukaryotic RNA” in Transcription and splicing (ed. B. D. Hames and D. M. Glover); Proudfoot (1989) Trends Biochem. Sci. 14: 105). These sequences direct the transcription of an mRNA which can be translated into the polypeptide encoded by the DNA. Examples of transcription terminator/polyadenylation signals include those derived from SV40 (Sambrook et al (1989) “Expression of cloned genes in cultured mammalian cells.” In Molecular Cloning: A Laboratory Manual). Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 (Gluzman (1981) Cell 23: 175) or polyomavirus, replicate to extremely high copy number in the presence of the appropriate viral T antigen. Additional examples of mammalian replicons include those derived from bovine papillomavirus and Epstein-Barr virus. Additionally, the replicon may have two replication systems, thus allowing it to be maintained, for example, in mammalian cells for expression and in a prokaryotic host for cloning and amplification. Examples of such mammalian-bacteria shuttle vectors include pMT2 (Kaufman et al. (1989) Mol. Cell. Biol. 9: 9469) and pHEBO (Shimizu et al. (1986) Mol. Cell. Biol. 6: 1074). The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide (s) in liposomes, and direct microinjection of the DNA into nuclei. Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g. Hep G2), and a number of other cell lines.

### Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media. After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence, and transcription termination sequence, are usually assembled into an intermediate transplacement construct (transfer vector). This may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extra-chromosomal element (e.g. plasmids) capable of stable maintenance in a host, such as a bacterium. The replicon will have a replication system, thus allowing it to be maintained in a suitable host for cloning and amplification. Currently, the most commonly used transfer vector for introducing foreign genes into AcNPV is pAc373. Many other vectors, known to those of skill in the art, have also been designed. These include, for example, pVL985 (which alters the polyhedrin start codon from ATG to AU, and which introduces a BamHI cloning site 32 basepairs downstream from the AU; see Luckow and Summers, Virology (1989) 17:31. The plasmid usually also contains the polyhedrin polyadenylation signal (Miller et al. (1988) Ann. Rev. Microbiol., 42: 177) and a prokaryotic ampicillin-resistance (amp) gene and origin of replication for selection and propagation in E. coli. Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765. DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human-interferon, Maeda et al., (1985), Nature 315:592; human gastrin-releasing peptide, Lebacq-Verheyden et al., (1988), Molec. Cell. Biol. 8: 3129; human IL-2, Smith et al., (1985) Proc. Natl. Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects. A recombinant polypeptide or polyprotein may be expressed intracellularly or, if it is expressed with the proper regulatory sequences, it can be secreted. Good intracellular expression of non-fused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3: 2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4: 91. The DNA sequence, when cloned in place of the polyhedrin gene in the expression vector, is flanked both 5′ and 3′ by polyhedrin-specific sequences and is positioned downstream of the polyhedrin promoter. The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 m in size, are highly retractile, giving them a bright shiny appearance that is readily visualized under the light microscope. Cells infected with recombinant viruses lack occlusion bodies. To distinguish recombinant virus from wild-type virus, the transfection supernatant is plagued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers and Smith, supra; Miller et al. (1989). Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56: 153; Wright (1986) Nature 321: 718; Smith et al., (1983) Mol. Cell. Biol. 3: 2156; and see generally, Fraser, et al. (1989) In Vitro Cell. Dev. Biol. 25: 225). Cells and cell culture media are commercially available for both direct and fusion expression of heterologous polypeptides in a baculovirus/expression system; cell culture technology is generally known to those skilled in the art. See, e.g. Summers and Smith supra. The modified insect cells may then be grown in an appropriate nutrient medium, which allows for stable maintenance of the plasmid (s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g. HPLC, affinity chromatography, ion exchange chromatography, etc.; electrophoresis; density gradient centrifugation; solvent extraction, etc. As appropriate, the product may be further purified, as required, so as to remove substantially any insect proteins which are also present in the medium, so as to provide a product which is at least substantially free of host debris, e.g. proteins, lipids and polysaccharides. In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

### Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30: 3861-3863 (1991). Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11 (2):165-185. Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art. The nucleic acid molecules of the subject invention may be included into an expression cassette for expression of the protein (s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector. A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellarepithelium layer into the endosperm of the seed. While it is not required that the protein be secreted from the cells in which the protein is produced, this facilitates the isolation and purification of the recombinant protein. Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985. The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipidsurfaced bodies, Fraley, et al., Proc. Natl. Acad. Sci. USA, 79, 1859-1863, 1982. The vector may also be introduced into the plant cells by electroporation. (Fromm et al., Proc. Natl Acad. Sci. USA 82: 5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus. All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura. Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

Preferred molecules according to the invention are disclosed in Tables 1 to 7 below.

In one embodiment the following tags are added at the C-terminus of the lysosomal proteins (see Table 1). Ideally, they contain linker sequences between the lysosomal protein and the tag. The C-terminal tags are fused with the lysosomal proteins in such a way that the tags replace the stop codon of the lysosomal proteins. In case C-terminal amino acids are omitted it is indicated.

TABLE 1
Antp and CLOCK

| | | |
|---|---|---|
| Linker | AGATCCCCCGGG | SEQ ID NO. 15 |
| preceding | | |
| the tags | | |
| “Antp” and | | |
| “CLOCK” | | |
| Linker | GGATCCCCCGGG | SEQ ID NO. 16 |
| preceding | | |
| the tags | | |
| “Antp” and | | |
| “CLOCK” | | |
| Antp cDNA | CGCCAGATAAAGATTTGGTTCCAGAATCGGCG | SEQ ID NO. 17 |
| of tag; | CATGAAGTGGAAGAAGTAA | |
| Antp amino | RQIKIWFQNRRMKWKK | SEQ ID NO. 18 |
| acid | | |
| sequence; | | |
| Human CLOCK | AAAAGAGTATCTAGAAACAAATCTGAAAAGAA | SEQ ID NO. 19 |
| cDNA tag; | ACGTAGATAA | |
| Human CLOCK | KRVSRNKSEKKRR | SEQ ID NO. 20 |
| amino acid | | |
| sequence | | |
| tag; | | |

The following tags are derived from the human basic fibroblast growth factor (FGF2) and possess an N-terminal linker (AGATCCGTCGACATCGAAGGTAGAGGCATT (SEQ ID NO. 21) or GGATCCGTCGACATCGAAGGTAGAGGCATT (SEQ ID NO. 23)) containing the factor Xa cleavage site “IEGR” (Table 2). The N-terminal linker may be mutated within the Xa cleavage site (IEGR) so that a base change through a mutation at by 24 of SEQ ID NO. 21 or 22 eliminates the factor Xa cleavage site “IEGR” by replacing the “R” by “S”. In context of the present invention the sequences within the fusion proteins encoded by a nucleic acid sequence according to SEQ ID NO. 21 or SEQ ID NO. 22 may be exchanged by a peptide sequence encoded by a nucleic acid sequence according to SEQ ID NO. 73 or SEQ ID NO. 74, respectively. Furthermore, the sequences according to SEQ ID NO. 21 or SEQ ID NO. 23 within the nucleotide sequences according to the present invention may be exchanged by a nucleotide sequence according to SEQ ID NO. 73 or SEQ ID NO. 74, respectively.

TABLE 2
FGF2 and variants thereof

| | | |
|---|---|---|
| N-terminal | AGATCCGTCGACATCGAAGGTAGAGGCATT | SEQ ID NO. 21 |
| linker | | |
| N-terminal | GGATCCGTCGACATCGAAGGTAGAGGCATT | SEQ ID NO. 22 |
| linker | | |
| N-terminal | AGATCCGTCGACATCGAAGGTAGCGGCATT | SEQ ID NO. 73 |
| linker with | | |
| mutated Xa | | |
| cleavage | | |
| site | | |
| N-terminal | GGATCCGTCGACATCGAAGGTAGCGGCATT | SEQ ID NO. 74 |
| linker with | | |
| mutated Xa | | |
| cleavage | | |
| site | | |
| FGF2 variant | CCCGCCTTGCCCGAGGATGGCGGCAGCGGCGC | SEQ ID NO. 23 |
| 1 (base | CTTCCCGCCCGGCCACTTCAAGGACCCCAAGC | |
| substitution | GGCTGTACTGCAAAAACGGGGGCTTCTTCCTG | |
| G206C and | CGCATCCACCCCGACGGCCGAGTTGACGGGGT | |
| G260C (small | CCGGGAGAAGAGCGACCCTCACATCAAGCTAC | |
| letter) | AACTTCAAGCAGAAGAGAGAGGAGTTGTGTCT | |
| leading to | ATCAAAGGAGTGTcTGCTAACCGTTACCTGGC | |
| amino acid | TATGAAGGAAGATGGAAGATTACTGGCTTCTA | |
| substitution | AATcTGTTACGGATGAGTGTTTCTTTTTTGAA | |
| s C69S and | CGATTGGAATCTAATAACTACAATACTTACCG | |
| C87S) | GTCAAGGAAATACACCAGTTGGTATGTGGCAC | |
| CDNA: | TGAAACGAACTGGGCAGTATAAACTTGGCTCC | |
| | AAAACAGGACCTGGGCAGAAAGCTATACTTTT | |
| | TCTTCCAATGTCTGCTAAGAGCTGA | |
| Amino acid | PALPEDGGSGAFPPGHFKDPKRLYCKNGGFFL | SEQ ID NO. 24 |
| sequence of | RIHPDGRVDGVREKSDPHIKLQLQAEERGVVS | |
| FGF2 variant | IKGVSANRYLAMKEDGRLLASKSVTDECFFFE | |
| 1 (base | RLESNNYNTYRSRKYTSWYVALKRTGQYKLGS | |
| substitution | KTGPGQKAILFLPMSAK | |
| G206C and | | |
| G260C (small | | |
| letter) | | |
| leading to | | |
| amino acid | | |
| substitution | | |
| s C69S and | | |
| C87S) | | |
| FGF2 variant | CCCGCCTTGCCCGAGGATGGCGGCAGCGGCGC | SEQ ID NO. 25 |
| 2 (same as | CTTCCCGCCCGGCCACTTCAAGGACCCCAAGC | |
| variant 1 | GGCTGTACTGCAAAAACGGGGGCTTCTTCCTG | |
| plus reduced | CGCATCCACCCCGACGGCCGAGTTGACGGGGT | |
| FGFR binding) | CCGGGAGAAGAGCGACCCTCACATCAAGCTAC | |
| cDNA | AACTTCAAGCAGAAGAGAGAGGAGTTGTGTCT | |
| | ATCAAAGGAGTGTCTGCTAACCGTTACCTGGC | |
| | TATGAAGGAAGATGGAAGATTACTGGCTTCTA | |
| | AATCTGTTACGGATGAGTGTTTCTTTTTTGCA | |
| | CGATTGGAATCTAATAACTACAATACTTACCG | |
| | GTCAAGGAAATACACCAGTTGGTATGTGGCAC | |
| | TGAAACGAACTGGGCAGTATAAACTTGGCTCC | |
| | AAAACAGGACCTGGGCAGAAAGCTATACTTTT | |
| | TCTTCCAATGTCTGCTAAGAGCTGA | |
| FGF2 variant | PALPEDGGSGAFPPGHFKDPKRLYCKNGGFFL | SEQ ID NO. 26 |
| 2 amino acid | RIHPDGRVDGVREKSDPHIKLQLQAEERGVVS | |
| sequence; | IKGVSANRYLAMKEDGRLLASKSVTDECFFFA | |
| | RLESNNYNTYRSRKYTSWYVALKRTGQYKLGS | |
| | KTGPGQKAILFLPMSAKS | |
| FGF2 variant | CCCGCCTTGCCCGAGGATGGCGGCAGCGGCGC | SEQ ID NO. 27 |
| 3 (same as | CTTCCCGCCCGGCCACTTCAAGGACCCCAAGC | |
| variant 1 | GGCTGTACTGCAAAAACGGGGGCTTCTTCCTG | |
| plus reduced | CGCATCCACCCCGACGGCCGAGTTGACGGGAC | |
| nuclear | AAGGGACAGGAGCGACCAGCACATTCAGCTGC | |
| translocation) | AGCTCAGTGCAGAAGAGAGAGGAGTTGTGTCT | |
| cDNA; | ATCAAAGGAGTGTCTGCTAACCGTTACCTGGC | |
| | TATGAAGGAAGATGGAAGATTACTGGCTTCTA | |
| | AATCTGTTACGGATGAGTGTTTCTTTTTTGAA | |
| | CGATTGGAATCTAATAACTACAATACTTACCG | |
| | GTCAAGGAAATACACCAGTTGGTATGTGGCAC | |
| | TGAAACGAACTGGGCAGTATAAACTTGGCTCC | |
| | AAAACAGGACCTGGGCAGAAAGCTATACTTTT | |
| | TCTTCCAATGTCTGCTAAGAGCTGA | |
| FGF2 variant | PALPEDGGSGAFPPGHFKDPKRLYCKNGGFFL | SEQ ID NO. 28 |
| 3 amino acid | RIHPDGRVDGTRDRSDQHIQLQLSAEERGVVS | |
| sequence; | IKGVSANRYLAMKEDGRLLASKSVTDECFFFE | |
| | RLESNNYNTYRSRKYTSWYVALKRTGQYKLGS | |
| | KTGPGQKAILFLPMSAKS | |
| FGF2 variant | CCCGCCTTGCCCGAGGATGGCGGCAGCGGCGC | SEQ ID NO. 29 |
| 4 (same as | CTTCCCGCCCGGCCACTTCAAGGACCCCAAGC | |
| variant 1 | GGCTGTACTGCAAAAACGGGGGCTTCTTCCTG | |
| plus reduced | CGCATCCACCCCGACGGCCGAGTTGACGGGAC | |
| FGFR binding | AAGGGACAGGAGCGACCAGCACATTCAGCTGC | |
| and reduced | AGCTCAGTGCAGAAGAGAGAGGAGTTGTGTCT | |
| nuclear | ATCAAAGGAGTGTCTGCTAACCGTTACCTGGC | |
| translocation) | TATGAAGGAAGATGGAAGATTACTGGCTTCTA | |
| cDNA: | AATCTGTTACGGATGAGTGTTTCTTTTTTGCA | |
| | CGATTGGAATCTAATAACTACAATACTTACCG | |
| | GTCAAGGAAATACACCAGTTGGTATGTGGCAC | |
| | TGAAACGAACTGGGCAGTATAAACTTGGCTCC | |
| | AAAACAGGACCTGGGCAGAAAGCTATACTTTT | |
| | TCTTCCAATGTCTGCTAAGAGCTGA | |
| FGF2 variant | PALPEDGGSGAFPPGHFKDPKRLYCKNGGFFL | SEQ ID NO. 30 |
| 4 amino acid | RIHPDGRVDGTRDRSDQHIQLQLSAEERGVVS | |
| sequence | IKGVSANRYLAMKEDGRLLASKSVTDECFFFA | |
| | RLESNNYNTYRSRKYTSWYVALKRTGQYKLGS | |
| | KTGPGQKAILFLPMSAKS | |

The following sequences demonstrate the C-terminal tags fused to the cDNA of human tripeptidyl peptidase 1 (TPP1) (Table 3).

TABLE 3
TPP1/Antp, TPP1/CLOCK and TPPI/FGF2 and variants
thereof

| | | |
|---|---|---|
| TPP1-Antp | ATGGGACTCCAAGCCTGCCTCCTAGGGCTCTT | SEQ ID NO. 31 |
| construct | TGCCCTCATCCTCTCTGGCAAATGCAGTTACA | |
| cDNA; | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCC | |
| | CCGGGCGCCAGATAAAGATTTGGTTCCAGAAT | |
| | CGGCGCATGAAGTGGAAGAAGTAA | |
| TPP1-Antp | MGLQACLLGLFALILSGKCSYSPEPDQRRTLP | SEQ ID NO. 32 |
| amino acid | PGWVSLGRADPEEELSLTFALRQQNVERLSEL | |
| sequence; | VQAVSDPSSPQYGKYLTLENVADLVRPSPLTL | |
| | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSPGRQIKIWFQN | |
| | RRMKWKK | |
| TPP1-CLOCK | ATGGGACTCCAAGCCTGCCTCCTAGGGCTCTT | SEQ ID NO. 33 |
| cDNA: | TGCCCTCATCCTCTCTGGCAAATGCAGTTACA | |
| | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCC | |
| | CCGGGAAAAGAGTATCTAGAAACAAATCTGAA | |
| | AAGAAACGTAGATAA | |
| TPP1-CLOCK | MGLQACLLGLFALILSGKCSYSPEPDQRRTLP | SEQ ID NO. 34 |
| amino acid | PGWVSLGRADPEEELSLTFALRQQNVERLSEL | |
| sequence: | VQAVSDPSSPQYGKYLTLENVADLVRPSPLTL | |
| | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSPGKRVSRNKSE | |
| | KKRR | |
| TPP1-FGF2 | ATGGGACTCCAAGCCTGCCTCCTAGGGCTCTT | SEQ ID NO. 35 |
| variant 1 | TGCCCTCATCCTCTCTGGCAAATGCAGTTACA | |
| cDNA: | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCG | |
| | TCGACATCGAAGGTAGAGGCATTCCCGCCTTG | |
| | CCCGAGGATGGCGGCAGCGGCGCCTTCCCGCC | |
| | CGGCCACTTCAAGGACCCCAAGCGGCTGTACT | |
| | GCAAAAACGGGGGCTTCTTCCTGCGCATCCAC | |
| | CCCGACGGCCGAGTTGACGGGGTCCGGGAGAA | |
| | GAGCGACCCTCACATCAAGCTACAACTTCAAG | |
| | CAGAAGAGAGAGGAGTTGTGTCTATCAAAGGA | |
| | GTGTCTGCTAACCGTTACCTGGCTATGAAGGA | |
| | AGATGGAAGATTACTGGCTTCTAAATCTGTTA | |
| | CGGATGAGTGTTTCTTTTTTGAACGATTGGAA | |
| | TCTAATAACTACAATACTTACCGGTCAAGGAA | |
| | ATACACCAGTTGGTATGTGGCACTGAAACGAA | |
| | CTGGGCAGTATAAACTTGGCTCCAAAACAGGA | |
| | CCTGGGCAGAAAGCTATACTTTTTCTTCCAAT | |
| | GTCTGCTAAGAGCTGA | |
| TPP1-FGF2 | MGLQACLLGLFALILSGKCSYSPEPDQRRTLP | SEQ ID NO. 36 |
| variant 1 | PGWVSLGRADPEEELSLTFALRQQNVERLSEL | |
| amino acid | VQAVSDPSSPQYGKYLTLENVADLVRPSPLTL | |
| sequence; | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSVDIEGRGIPAL | |
| | PEDGGSGAFPPGHFKDPKRLYCKNGGFFLRIH | |
| | PDGRVDGVREKSDPHIKLQLQAEERGVVSIKG | |
| | VSANRYLAMKEDGRLLASKSVTDECFFFERLE | |
| | SNNYNTYRSRKYTSWYVALKRTGQYKLGSKTG | |
| | PGQKAILFLPMSAKS | |
| TPP1-FGF2 | ATGGGACTCCAAGCCTGCCTCCTAGGGCTCTT | SEQ ID NO. 37 |
| variant 2 | TGCCCTCATCCTCTCTGGCAAATGCAGTTACA | |
| cDNA; | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCG | |
| | TCGACATCGAAGGTAGAGGCATTCCCGCCTTG | |
| | CCCGAGGATGGCGGCAGCGGCGCCTTCCCGCC | |
| | CGGCCACTTCAAGGACCCCAAGCGGCTGTACT | |
| | GCAAAAACGGGGGCTTCTTCCTGCGCATCCAC | |
| | CCCGACGGCCGAGTTGACGGGGTCCGGGAGAA | |
| | GAGCGACCCTCACATCAAGCTACAACTTCAAG | |
| | CAGAAGAGAGAGGAGTTGTGTCTATCAAAGGA | |
| | GTGTCTGCTAACCGTTACCTGGCTATGAAGGA | |
| | AGATGGAAGATTACTGGCTTCTAAATCTGTTA | |
| | CGGATGAGTGTTTCTTTTTTGCACGATTGGAA | |
| | TCTAATAACTACAATACTTACCGGTCAAGGAA | |
| | ATACACCAGTTGGTATGTGGCACTGAAACGAA | |
| | CTGGGCAGTATAAACTTGGCTCCAAAACAGGA | |
| | CCTGGGCAGAAAGCTATACTTTTTCTTCCAAT | |
| | GTCTGCTAAGAGCTGA | |
| TPP1-FGF2 | MGLQACLLGLFALILSGKCSYSPEPDQRRTLP | SEQ ID NO. 38 |
| variant 2 | PGWVSLGRADPEEELSLTFALRQQNVERLSEL | |
| amino acid | VQAVSDPSSPQYGKYLTLENVADLVRPSPLTL | |
| sequence; | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSVDIEGRGIPAL | |
| | PEDGGSGAFPPGHFKDPKRLYCKNGGFFLRIH | |
| | PDGRVDGVREKSDPHIKLQLQAEERGVVSIKG | |
| | VSANRYLAMKEDGRLLASKSVTDECFFFARLE | |
| | SNNYNTYRSRKYTSWYVALKRTGQYKLGSKTG | |
| | PGQKAILFLPMSAKS | |
| TPP1-FGF2 | ATGGGACTCCAAGCCTGCCTCCTAGGGCTCTT | SEQ ID NO. 39 |
| variant 3 | TGCCCTCATCCTCTCTGGCAAATGCAGTTACA | |
| cDNA; | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCG | |
| | TCGACATCGAAGGTAGAGGCATTCCCGCCTTG | |
| | CCCGAGGATGGCGGCAGCGGCGCCTTCCCGCC | |
| | CGGCCACTTCAAGGACCCCAAGCGGCTGTACT | |
| | GCAAAAACGGGGGCTTCTTCCTGCGCATCCAC | |
| | CCCGACGGCCGAGTTGACGGGACAAGGGACAG | |
| | GAGCGACCAGCACATTCAGCTGCAGCTCAGTG | |
| | CAGAAGAGAGAGGAGTTGTGTCTATCAAAGGA | |
| | GTGTCTGCTAACCGTTACCTGGCTATGAAGGA | |
| | AGATGGAAGATTACTGGCTTCTAAATCTGTTA | |
| | CGGATGAGTGTTTCTTTTTTGAACGATTGGAA | |
| | TCTAATAACTACAATACTTACCGGTCAAGGAA | |
| | ATACACCAGTTGGTATGTGGCACTGAAACGAA | |
| | CTGGGCAGTATAAACTTGGCTCCAAAACAGGA | |
| | CCTGGGCAGAAAGCTATACTTTTTCTTCCAAT | |
| | GTCTGCTAAGAGCTGA | |
| TPP1-FGF2 | MGLQACLLGLFALILSGKCSYSPEPDQRRTLP | SEQ ID NO. 40 |
| variant 3 | PGWVSLGRADPEEELSLTFALRQQNVERLSEL | |
| amino acid | VQAVSDPSSPQYGKYLTLENVADLVRPSPLTL | |
| sequence; | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSVDTEGRGIPAL | |
| | PEDGGSGAFPPGHFKDPKRLYCKNGGFFLRIH | |
| | PDGRVDGTRDRSDQHIQLQLSAEERGVVSIKG | |
| | VSANRYLAMKEDGRLLASKSVTDECFFFERLE | |
| | SNNYNTYRSRKYTSWYVALKRTGQYKLGSKTG | |
| | PGQKAILFLPMSAKS | |
| TPP1-FGF2 | ATGGGACTCCAAGCCTGCCTCCTAGGGCTCTT | SEQ ID NO. 41 |
| variant 4 | TGCCCTCATCCTCTCTGGCAAATGCAGTTACA | |
| cDNA; | GCCCGGAGCCCGACCAGCGGAGGACGCTGCCC | |
| | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCAGATCCG | |
| | TCGACATCGAAGGTAGAGGCATTCCCGCCTTG | |
| | CCCGAGGATGGCGGCAGCGGCGCCTTCCCGCC | |
| | CGGCCACTTCAAGGACCCCAAGCGGCTGTACT | |
| | GCAAAAACGGGGGCTTCTTCCTGCGCATCCAC | |
| | CCCGACGGCCGAGTTGACGGGACAAGGGACAG | |
| | GAGCGACCAGCACATTCAGCTGCAGCTCAGTG | |
| | CAGAAGAGAGAGGAGTTGTGTCTATCAAAGGA | |
| | GTGTCTGCTAACCGTTACCTGGCTATGAAGGA | |
| | AGATGGAAGATTACTGGCTTCTAAATCTGTTA | |
| | CGGATGAGTGTTTCTTTTTTGCACGATTGGAA | |
| | TCTAATAACTACAATACTTACCGGTCAAGGAA | |
| | ATACACCAGTTGGTATGTGGCACTGAAACGAA | |
| | CTGGGCAGTATAAACTTGGCTCCAAAACAGGA | |
| | CCTGGGCAGAAAGCTATACTTTTTCTTCCAAT | |
| | GTCTGCTAAGAGCTGA | |
| TPP1-FGF2 | MGLQACLLGLFALILSGKCSYSPEPDQRRTLP | SEQ ID NO. 42 |
| variant 4 | PGWVSLGRADPEEELSLTFALRQQNVERLSEL | |
| amino acid | VQAVSDPSSPQYGKYLTLENVADLVRPSPLTL | |
| sequence; | HTVQKWLLAAGAQKCHSVITQDFLTCWLSIRQ | |
| | AELLLPGAEFHHYVGGPTETHVVRSPHPYQLP | |
| | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNPRSVDIEGRGIPAL | |
| | PEDGGSGAFPPGHFKDPKRLYCKNGGFFLRIH | |
| | PDGRVDGTRDRSDQHIQLQLSAEERGVVSIKG | |
| | VSANRYLAMKEDGRLLASKSVTDECFFFARLE | |
| | SNNYNTYRSRKYTSWYVALKRTGQYKLGSKTG | |
| | PGQKAILFLPMSAKS | |

The following tags (Table 4) are derived from the human heparin-binding epidermal growth factor (HB-EGF). They are added at the N-terminus of the lysosomal proteins and replace the signal peptide of the lysosomal proteins.

Two different HB-EGF tags were designed. The last nucleotide “T” of HB1 and HB2 is alternatively replaced by “C”:

TABLE 4
HB1 and HB2 tags

| | | |
|---|---|---|
| HB1 cDNA | ATGCAGCCCTCCAGCCTTCTGCCGCTCGCCCT | SEQ ID NO. 43 |
| | CTGCCTGCTGGCTGCACCCGCCGGATCTTCCA | |
| | AGCCACAAGCACTGGCCACACCAAACAAGGAG | |
| | GAGCACGGGAAAAGAAAGAAGAAAGGCAAGGG | |
| | GCTAGGGAAGAAGAGGGACCCATGTCTTCGGA | |
| | AATACAAGGACTTCTGCATCCATGGAGAATGC | |
| | AAATATGTGAAGGAGCTCCGGGCTCCCTCCTG | |
| | CATCTGCCACCCGGGTTACCATGGAGAGAGGT | |
| | GTCATGGGCTGAGCGGATCT | |
| HB2 cDNA | ATGCAGCCCTCCAGCCTTCTGCCGCTCGCCCT | SEQ ID NO. 44 |
| | CTGCCTGCTGGCTGCACCCGCCGGATCTGGGA | |
| | AAAGAAAGAAGAAAGGCAAGGGGCTAGGGAAG | |
| | AAGAGGGACCCATCTCTTCGGAAATACAAGGA | |
| | CTTCTCCGGATCT | |
| HB1 amino | MQPSSLLPLALCLLAAPAGSSKPQALATPNKE | SEQ ID NO. 71 |
| acid | EHGKRKKKGKGLGKKRDPCLRKYKDFCIHGEC | |
| sequence | KYVKELRAPSCICHPGYHGERCHGLSGS | |
| HB2 amino | MQPSSLLPLALCLLAAPAGSGKRKKKGKGLGK | SEQ ID NO. 72 |
| acid | KRDPSLRKYKDFSGS | |
| sequence | | |

The following sequences (Table 5) disclose the N-terminal tags fused to the cDNA of human sulfamidase (hSGSH)

TABLE 5
HB1/SGSH and HB2/SGSH

| | | |
|---|---|---|
| HB1-SGSH | ATGCAGCCCTCCAGCCTTCTGCCGCTCGCCCT | SEQ ID NO. 45 |
| cDNA: | CTGCCTGCTGGCTGCACCCGCCGGATCTTCCA | |
| | AGCCACAAGCACTGGCCACACCAAACAAGGAG | |
| | GAGCACGGGAAAAGAAAGAAGAAAGGCAAGGG | |
| | GCTAGGGAAGAAGAGGGACCCATGTCTTCGGA | |
| | AATACAAGGACTTCTGCATCCATGGAGAATGC | |
| | AAATATGTGAAGGAGCTCCGGGCTCCCTCCTG | |
| | CATCTGCCACCCGGGTTACCATGGAGAGAGGT | |
| | GTCATGGGCTGAGCGGATCTCGTCCCCGGAAC | |
| | GCACTGCTGCTCCTCGCGGATGACGGAGGCTT | |
| | TGAGAGTGGCGCGTACAACAACAGCGCCATCG | |
| | CCACCCCGCACCTGGACGCCTTGGCCCGCCGC | |
| | AGCCTCCTCTTTCGCAATGCCTTCACCTCGGT | |
| | CAGCAGCTGCTCTCCCAGCCGCGCCAGCCTCC | |
| | TCACTGGCCTGCCCCAGCATCAGAATGGGATG | |
| | TACGGGCTGCACCAGGACGTGCACCACTTCAA | |
| | CTCCTTCGACAAGGTGCGGAGCCTGCCGCTGC | |
| | TGCTCAGCCAAGCTGGTGTGCGCACAGGCATC | |
| | ATCGGGAAGAAGCACGTGGGGCCGGAGACCGT | |
| | GTACCCGTTTGACTTTGCGTACACGGAGGAGA | |
| | ATGGCTCCGTCCTCCAGGTGGGGCGGAACATC | |
| | ACTAGAATTAAGCTGCTCGTCCGGAAATTCCT | |
| | GCAGACTCAGGATGACCGGCCTTTCTTCCTCT | |
| | ACGTCGCCTTCCACGACCCCCACCGCTGTGGG | |
| | CACTCCCAGCCCCAGTACGGAACCTTCTGTGA | |
| | GAAGTTTGGCAACGGAGAGAGCGGCATGGGTC | |
| | GTATCCCAGACTGGACCCCCCAGGCCTACGAC | |
| | CCACTGGACGTGCTGGTGCCTTACTTCGTCCC | |
| | CAACACCCCGGCAGCCCGAGCCGACCTGGCCG | |
| | CTCAGTACACCACCGTAGGCCGCATGGACCAA | |
| | GGAGTTGGACTGGTGCTCCAGGAGCTGCGTGA | |
| | CGCCGGTGTCCTGAACGACACACTGGTGATCT | |
| | TCACGTCCGACAACGGGATCCCCTTCCCCAGC | |
| | GGCAGGACCAACCTGTACTGGCCGGGCACTGC | |
| | TGAACCCTTACTGGTGTCATCCCCGGAGCACC | |
| | CAAAACGCTGGGGCCAAGTCAGCGAGGCCTAC | |
| | GTGAGCCTCCTAGACCTCACGCCCACCATCTT | |
| | GGATTGGTTCTCGATCCCGTACCCCAGCTACG | |
| | CCATCTTTGGCTCGAAGACCATCCACCTCACT | |
| | GGCCGGTCCCTCCTGCCGGCGCTGGAGGCCGA | |
| | GCCCCTCTGGGCCACCGTCTTTGGCAGCCAGA | |
| | GCCACCACGAGGTCACCATGTCCTACCCCATG | |
| | CGCTCCGTGCAGCACCGGCACTTCCGCCTCGT | |
| | GCACAACCTCAACTTCAAGATGCCCTTTCCCA | |
| | TCGACCAGGACTTCTACGTCTCACCCACCTTC | |
| | CAGGACCTCCTGAACCGCACTACAGCTGGTCA | |
| | GCCCACGGGCTGGTACAAGGACCTCCGTCATT | |
| | ACTACTACCGGGCGCGCTGGGAGCTCTACGAC | |
| | CGGAGCCGGGACCCCCACGAGACCCAGAACCT | |
| | GGCCACCGACCCGCGCTTTGCTCAGCTTCTGG | |
| | AGATGCTTCGGGACCAGCTGGCCAAGTGGCAG | |
| | TGGGAGACCCACGACCCCTGGGTGTGCGCCCC | |
| | CGACGGCGTCCTGGAGGAGAAGCTCTCTCCCC | |
| | AGTGCCAGCCCCTCCACAATGAGCTGTAA | |
| HB1-SGSH | MQPSSLLPLALCLLAARAGSSKPQALATPNKE | SEQ ID NO. 46 |
| amino acid | EHGKRKKKGKGLGKKRDPCLRKYKDFCIHGEC | |
| sequence; | KYVKELRAPSCICHPGYHGERCHGLSGSRPRN | |
| | ALLLLADDGGFESGAYNNSAIATPHLDALARR | |
| | SLLFRNAFTSVSSCSPSRASLLTGLPQHQNGM | |
| | YGLHQDVHHFNSFDKVRSLPLLLSQAGVRTGI | |
| | IGKKHVGPETVYPFDFAYTEENGSVLQVGRNI | |
| | TRIKLLVRKFLQTQDDRPFFLYVAFHDPHRCG | |
| | HSQPQYGTFCEKFGNGESGMGRIPDWTPQAYD | |
| | PLDVLVPYFVPNTPAARADLAAQYTTVGRMDQ | |
| | GVGLVLQELRDAGVLNDTLVIFTSDNGIPFPS | |
| | GRTNLYWPGTAEPLLVSSPEHPKRWGQVSEAY | |
| | VSLLDLTPTILDWFSIPYPSYAIFGSKTIHLT | |
| | GRSLLPALEAEPLWATVFGSQSHHEVTMSYPM | |
| | RSVQHRHFRLVHNLNFKMPFPIDQDFYVSPTF | |
| | QDLLNRTTAGQPTGWYKDLRHYYYRARWELYD | |
| | RSRDPHETQNLATDPRFAQLLEMLRDQLAKWQ | |
| | WETHDPWVCAPDGVLEEKLSPQCQPLHNEL | |
| HB2-SGSH | ATGCAGCCCTCCAGCCTTCTGCCGCTCGCCCT | SEQ ID NO. 47 |
| cDNA: | CTGCCTGCTGGCTGCACCCGCCGGATCTGGGA | |
| | AAAGAAAGAAGAAAGGCAAGGGGCTAGGGAAG | |
| | AAGAGGGACCCATCTCTTCGGAAATACAAGGA | |
| | CTTCTCCGGATCTCGTCCCCGGAACGCACTGC | |
| | TGCTCCTCGCGGATGACGGAGGCTTTGAGAGT | |
| | GGCGCGTACAACAACAGCGCCATCGCCACCCC | |
| | GCACCTGGACGCCTTGGCCCGCCGCAGCCTCC | |
| | TCTTTCGCAATGCCTTCACCTCGGTCAGCAGC | |
| | TGCTCTCCCAGCCGCGCCAGCCTCCTCACTGG | |
| | CCTGCCCCAGCATCAGAATGGGATGTACGGGC | |
| | TGCACCAGGACGTGCACCACTTCAACTCCTTC | |
| | GACAAGGTGCGGAGCCTGCCGCTGCTGCTCAG | |
| | CCAAGCTGGTGTGCGCACAGGCATCATCGGGA | |
| | AGAAGCACGTGGGGCCGGAGACCGTGTACCCG | |
| | TTTGACTTTGCGTACACGGAGGAGAATGGCTC | |
| | CGTCCTCCAGGTGGGGCGGAACATCACTAGAA | |
| | TTAAGCTGCTCGTCCGGAAATTCCTGCAGACT | |
| | CAGGATGACCGGCCTTTCTTCCTCTACGTCGC | |
| | CTTCCACGACCCCCACCGCTGTGGGCACTCCC | |
| | AGCCCCAGTACGGAACCTTCTGTGAGAAGTTT | |
| | GGCAACGGAGAGAGCGGCATGGGTCGTATCCC | |
| | AGACTGGACCCCCCAGGCCTACGACCCACTGG | |
| | ACGTGCTGGTGCCTTACTTCGTCCCCAACACC | |
| | CCGGCAGCCCGAGCCGACCTGGCCGCTCAGTA | |
| | CACCACCGTAGGCCGCATGGACCAAGGAGTTG | |
| | GACTGGTGCTCCAGGAGCTGCGTGACGCCGGT | |
| | GTCCTGAACGACACACTGGTGATCTTCACGTC | |
| | CGACAACGGGATCCCCTTCCCCAGCGGCAGGA | |
| | CCAACCTGTACTGGCCGGGCACTGCTGAACCC | |
| | TTACTGGTGTCATCCCCGGAGCACCCAAAACG | |
| | CTGGGGCCAAGTCAGCGAGGCCTACGTGAGCC | |
| | TCCTAGACCTCACGCCCACCATCTTGGATTGG | |
| | TTCTCGATCCCGTACCCCAGCTACGCCATCTT | |
| | TGGCTCGAAGACCATCCACCTCACTGGCCGGT | |
| | CCCTCCTGCCGGCGCTGGAGGCCGAGCCCCTC | |
| | TGGGCCACCGTCTTTGGCAGCCAGAGCCACCA | |
| | CGAGGTCACCATGTCCTACCCCATGCGCTCCG | |
| | TGCAGCACCGGCACTTCCGCCTCGTGCACAAC | |
| | CTCAACTTCAAGATGCCCTTTCCCATCGACCA | |
| | GGACTTCTACGTCTCACCCACCTTCCAGGACC | |
| | TCCTGAACCGCACTACAGCTGGTCAGCCCACG | |
| | GGCTGGTACAAGGACCTCCGTCATTACTACTA | |
| | CCGGGCGCGCTGGGAGCTCTACGACCGGAGCC | |
| | GGGACCCCCACGAGACCCAGAACCTGGCCACC | |
| | GACCCGCGCTTTGCTCAGCTTCTGGAGATGCT | |
| | TCGGGACCAGCTGGCCAAGTGGCAGTGGGAGA | |
| | CCCACGACCCCTGGGTGTGCGCCCCCGACGGC | |
| | GTCCTGGAGGAGAAGCTCTCTCCCCAGTGCCA | |
| | GCCCCTCCACAATGAGCTGTAA | |
| HB2-SGSH | MQPSSLLPLALCLLAAPAGSGKRKKKGKGLGK | SEQ ID NO. 48 |
| amino acid | KRDPSLRKYKDFSGSRPRNALLLLADDGGFES | |
| sequence; | GAYNNSAIATPHLDALARRSLLFRNAFTSVSS | |
| | CSPSRASLLTGLPQHQNGMYGLHQDVHHFNSF | |
| | DKVRSLPLLLSQAGVRTGIIGKKHVGPETVYP | |
| | FDFAYTEENGSVLQVGRNITRIKLLVRKFLQT | |
| | QDDRPFFLYVAFHDPHRCGHSQPQYGTFCEKF | |
| | GNGESGMGRIPDWTPQAYDPLDVLVPYFVPNT | |
| | PAARADLAAQYTTVGRMDQGVGLVLQELRDAG | |
| | VLNDTLVIFTSDNGIPFPSGRTNLYWPGTAEP | |
| | LLVSSPEHPKRWGQVSEAYVSLLDLTPTILDW | |
| | FSIPYPSYAIFGSKTIHLTGRSLLPALEAEPL | |
| | WATVFGSQSHHEVTMSYPMRSVQHRHFRLVHN | |
| | LNFKMPFPIDQDFYVSPTFQDLLNRTTAGQPT | |
| | GWYKDLRHYYYRARWELYDRSRDPHETQNLAT | |
| | DPRFAQLLEMLRDQLAKWQWETHDPWVCAPDG | |
| | VLEEKLSPQCQPLHNEL | |

Combined N-terminal and C-terminal heparin/heparan sulfate binding tags were constructed correspondingly. The combined N-terminal and C-terminal tag is demonstrated for human sulfamidase (SGSH) (Table 6).

TABLE 6
HB2/SGSH/Antp

| | | |
|---|---|---|
| HB2-SGSH-Antp | ATGCAGCCCTCCAGCCTTCTGCCGCTCGCCCT | SEQ ID NO. 49 |
| cDNA; | CTGCCTGCTGGCTGCACCCGCCGGATCTGGGA | |
| | AAAGAAAGAAGAAAGGCAAGGGGCTAGGGAAG | |
| | AAGAGGGACCCATCTCTTCGGAAATACAAGGA | |
| | CTTCTCCGGATCTCGTCCCCGGAACGCACTGC | |
| | TGCTCCTCGCGGATGACGGAGGCTTTGAGAGT | |
| | GGCGCGTACAACAACAGCGCCATCGCCACCCC | |
| | GCACCTGGACGCCTTGGCCCGCCGCAGCCTCC | |
| | TCTTTCGCAATGCCTTCACCTCGGTCAGCAGC | |
| | TGCTCTCCCAGCCGCGCCAGCCTCCTCACTGG | |
| | CCTGCCCCAGCATCAGAATGGGATGTACGGGC | |
| | TGCACCAGGACGTGCACCACTTCAACTCCTTC | |
| | GACAAGGTGCGGAGCCTGCCGCTGCTGCTCAG | |
| | CCAAGCTGGTGTGCGCACAGGCATCATCGGGA | |
| | AGAAGCACGTGGGGCCGGAGACCGTGTACCCG | |
| | TTTGACTTTGCGTACACGGAGGAGAATGGCTC | |
| | CGTCCTCCAGGTGGGGCGGAACATCACTAGAA | |
| | TTAAGCTGCTCGTCCGGAAATTCCTGCAGACT | |
| | CAGGATGACCGGCCTTTCTTCCTCTACGTCGC | |
| | CTTCCACGACCCCCACCGCTGTGGGCACTCCC | |
| | AGCCCCAGTACGGAACCTTCTGTGAGAAGTTT | |
| | GGCAACGGAGAGAGCGGCATGGGTCGTATCCC | |
| | AGACTGGACCCCCCAGGCCTACGACCCACTGG | |
| | ACGTGCTGGTGCCTTACTTCGTCCCCAACACC | |
| | CCGGCAGCCCGAGCCGACCTGGCCGCTCAGTA | |
| | CACCACCGTAGGCCGCATGGACCAAGGAGTTG | |
| | GACTGGTGCTCCAGGAGCTGCGTGACGCCGGT | |
| | GTCCTGAACGACACACTGGTGATCTTCACGTC | |
| | CGACAACGGGATCCCCTTCCCCAGCGGCAGGA | |
| | CCAACCTGTACTGGCCGGGCACTGCTGAACCC | |
| | TTACTGGTGTCATCCCCGGAGCACCCAAAACG | |
| | CTGGGGCCAAGTCAGCGAGGCCTACGTGAGCC | |
| | TCCTAGACCTCACGCCCACCATCTTGGATTGG | |
| | TTCTCGATCCCGTACCCCAGCTACGCCATCTT | |
| | TGGCTCGAAGACCATCCACCTCACTGGCCGGT | |
| | CCCTCCTGCCGGCGCTGGAGGCCGAGCCCCTC | |
| | TGGGCCACCGTCTTTGGCAGCCAGAGCCACCA | |
| | CGAGGTCACCATGTCCTACCCCATGCGCTCCG | |
| | TGCAGCACCGGCACTTCCGCCTCGTGCACAAC | |
| | CTCAACTTCAAGATGCCCTTTCCCATCGACCA | |
| | GGACTTCTACGTCTCACCCACCTTCCAGGACC | |
| | TCCTGAACCGCACTACAGCTGGTCAGCCCACG | |
| | GGCTGGTACAAGGACCTCCGTCATTACTACTA | |
| | CCGGGCGCGCTGGGAGCTCTACGACCGGAGCC | |
| | GGGACCCCCACGAGACCCAGAACCTGGCCACC | |
| | GACCCGCGCTTTGCTCAGCTTCTGGAGATGCT | |
| | TCGGGACCAGCTGGCCAAGTGGCAGTGGGAGA | |
| | CCCACGACCCCTGGGTGTGCGCCCCCGACGGC | |
| | GTCCTGGAGGAGAAGCTCTCTCCCCAGTGCCA | |
| | GCCCCTCCACAATGAGCTGAGATCCCCCGGGC | |
| | GCCAGATAAAGATTTGGTTCCAGAATCGGCGC | |
| | ATGAAGTGGAAGAAGTAA | |
| HB2-SGSH-Antp | MQPSSLLPLALCLLAAPAGSGKRKKKGKGLGK | SEQ ID NO. 50 |
| amino acid | KRDPSLRKYKDFSGSRPRNALLLLADDGGFES | |
| sequence | GAYNNSAIATPHLDALARRSLLFRNAFTSVSS | |
| | CSPSRASLLTGLPQHQNGMYGLHQDVHHFNSF | |
| | DKVRSLPLLLSQAGVRTGIIGKKHVGPETVYP | |
| | FDFAYTEENGSVLQVGRNITRIKLLVRKFLQT | |
| | QDDRPFFLYVAFHDPHRCGHSQPQYGTFCEKF | |
| | GNGESGMGRIPDWTPQAYDPLDVLVPYFVPNT | |
| | PAARADLAAQYTTVGRMDQGVGLVLQELRDAG | |
| | VLNDTLVIFTSDNGIPFPSGRTNLYWPGTAEP | |
| | LLVSSPEHPKRWGQVSEAYVSLLDLTPTILDW | |
| | FSIPYPSYAIFGSKTIHLTGRSLLPALEAEPL | |
| | WATVFGSQSHHEVTMSYPMRSVQHRHFRLVHN | |
| | LNFKMPFPIDQDFYVSPTFQDLLNRTTAGQPT | |
| | GWYKDLRHYYYRARWELYDRSRDPHETQNLAT | |
| | DPRFAQLLEMLRDOLAKWQWETHDPWVCAPDG | |
| | VLEEKLSPQCQPLHNELRSPGRQIKIWFQNRR | |
| | MKWKK | |

The following lysosomal proteins were fused to the above described heparin/heparin sulfate binding tags (Table 7).

TABLE 7
TPP1, CTSD, PPT1, SGSH, IDUA, IDS, ARSA, GALC, GBA and
GLA

| | | |
|---|---|---|
| Human | ATGGGACTCCAAGCCTGCCTCCTAGGGCTCTT | SEQ ID NO. 51 |
| tripeptidyl | TGCCCTCATCCTCTCTGGCAAATGCAGTTACA | |
| peptidase 1 | GCCCGGAGCCCGACCAGCGGAGGACGCTGCCC | |
| (TPP1) cDNA: | CCAGGCTGGGTGTCCCTGGGCCGTGCGGACCC | |
| | TGAGGAAGAGCTGAGTCTCACCTTTGCCCTGA | |
| | GACAGCAGAATGTGGAAAGACTCTCGGAGCTG | |
| | GTGCAGGCTGTGTCGGATCCCAGCTCTCCTCA | |
| | ATACGGAAAATACCTGACCCTAGAGAATGTGG | |
| | CTGATCTGGTGAGGCCATCCCCACTGACCCTC | |
| | CACACGGTGCAAAAATGGCTCTTGGCAGCCGG | |
| | AGCCCAGAAGTGCCATTCTGTGATCACACAGG | |
| | ACTTTCTGACTTGCTGGCTGAGCATCCGACAA | |
| | GCAGAGCTGCTGCTCCCTGGGGCTGAGTTTCA | |
| | TCACTATGTGGGAGGACCTACGGAAACCCATG | |
| | TTGTAAGGTCCCCACATCCCTACCAGCTTCCA | |
| | CAGGCCTTGGCCCCCCATGTGGACTTTGTGGG | |
| | GGGACTGCACCGTTTTCCCCCAACATCATCCC | |
| | TGAGGCAACGTCCTGAGCCGCAGGTGACAGGG | |
| | ACTGTAGGCCTGCATCTGGGGGTAACCCCCTC | |
| | TGTGATCCGTAAGCGATACAACTTGACCTCAC | |
| | AAGACGTGGGCTCTGGCACCAGCAATAACAGC | |
| | CAAGCCTGTGCCCAGTTCCTGGAGCAGTATTT | |
| | CCATGACTCAGACCTGGCTCAGTTCATGCGCC | |
| | TCTTCGGTGGCAACTTTGCACATCAGGCATCA | |
| | GTAGCCCGTGTGGTTGGACAACAGGGCCGGGG | |
| | CCGGGCCGGGATTGAGGCCAGTCTAGATGTGC | |
| | AGTACCTGATGAGTGCTGGTGCCAACATCTCC | |
| | ACCTGGGTCTACAGTAGCCCTGGCCGGCATGA | |
| | GGGACAGGAGCCCTTCCTGCAGTGGCTCATGC | |
| | TGCTCAGTAATGAGTCAGCCCTGCCACATGTG | |
| | CATACTGTGAGCTATGGAGATGATGAGGACTC | |
| | CCTCAGCAGCGCCTACATCCAGCGGGTCAACA | |
| | CTGAGCTCATGAAGGCTGCCGCTCGGGGTCTC | |
| | ACCCTGCTCTTCGCCTCAGGTGACAGTGGGGC | |
| | CGGGTGTTGGTCTGTCTCTGGAAGACACCAGT | |
| | TCCGCCCTACCTTCCCTGCCTCCAGCCCCTAT | |
| | GTCACCACAGTGGGAGGCACATCCTTCCAGGA | |
| | ACCTTTCCTCATCACAAATGAAATTGTTGACT | |
| | ATATCAGTGGTGGTGGCTTCAGCAATGTGTTC | |
| | CCACGGCCTTCATACCAGGAGGAAGCTGTAAC | |
| | GAAGTTCCTGAGCTCTAGCCCCCACCTGCCAC | |
| | CATCCAGTTACTTCAATGCCAGTGGCCGTGCC | |
| | TACCCAGATGTGGCTGCACTTTCTGATGGCTA | |
| | CTGGGTGGTCAGCAACAGAGTGCCCATTCCAT | |
| | GGGTGTCCGGAACCTCGGCCTCTACTCCAGTG | |
| | TTTGGGGGGATCCTATCCTTGATCAATGAGCA | |
| | CAGGATCCTTAGTGGCCGCCCCCCTCTTGGCT | |
| | TTCTCAACCCAAGGCTCTACCAGCAGCATGGG | |
| | GCAGGACTCTTTGATGTAACCCGTGGCTGCCA | |
| | TGAGTCCTGTCTGGATGAAGAGGTAGAGGGCC | |
| | AGGGTTTCTGCTCTGGTCCTGGCTGGGATCCT | |
| | GTAACAGGCTGGGGAACACCCAACTTCCCAGC | |
| | TTTGCTGAAGACTCTACTCAACCCCTGA | |
| Human | MGLQACLLGLFALILSGKCSYSPEPDQRRTLP | SEQ ID NO. 52 |
| tripeptidyl | PGWVSLGRADPEEELSLTFALRQQNVERLSEL | |
| peptidase 1 | VQAVSDPSSPQYGKYLTLENVADLVRPSPLTL | |
| (TPP1) amino | HTVQKWLLAAGAQKCHSVITQDFLTCWLSIRQ | |
| acid | AELLLPGAEFHHYVGGPTETHVVRSPHPYQLP | |
| sequence; | QALAPHVDFVGGLHRFPPTSSLRQRPEPQVTG | |
| | TVGLHLGVTPSVIRKRYNLTSQDVGSGTSNNS | |
| | QACAQFLEQYFHDSDLAQFMRLFGGNFAHQAS | |
| | VARVVGQQGRGRAGIEASLDVQYLMSAGANIS | |
| | TWVYSSPGRHEGQEPFLQWLMLLSNESALPHV | |
| | HTVSYGDDEDSLSSAYIQRVNTELMKAAARGL | |
| | TLLFASGDSGAGCWSVSGRHQFRPTFPASSPY | |
| | VTTVGGTSFQEPFLITNEIVDYISGGGFSNVF | |
| | PRPSYQEEAVTKFLSSSPHLPPSSYFNASGRA | |
| | YPDVAALSDGYWVVSNRVPIPWVSGTSASTPV | |
| | FGGILSLINEHRILSGRPPLGFLNPRLYQQHG | |
| | AGLFDVTRGCHESCLDEEVEGQGFCSGPGWDP | |
| | VTGWGTPNFPALLKTLLNP | |
| Human | ATGCAGCCCTCCAGCCTTCTGCCGCTCGCCCT | SEQ ID NO. 53 |
| cathepsin D | CTGCCTGCTGGCTGCACCCGCCTCCGCGCTCG | |
| (CTSD) cDNA; | TCAGGATCCCGCTGCACAAGTTCACGTCCATC | |
| | CGCCGGACCATGTCGGAGGTTGGGGGCTCTGT | |
| | GGAGGACCTGATTGCCAAAGGCCCCGTCTCAA | |
| | AGTACTCCCAGGCGGTGCCAGCCGTGACCGAG | |
| | GGGCCCATTCCCGAGGTGCTCAAGAACTACAT | |
| | GGACGCCCAGTACTACGGGGAGATTGGCATCG | |
| | GGACGCCCCCCCAGTGCTTCACAGTCGTCTTC | |
| | GACACGGGCTCCTCCAACCTGTGGGTCCCCTC | |
| | CATCCACTGCAAACTGCTGGACATCGCTTGCT | |
| | GGATCCACCACAAGTACAACAGCGACAAGTCC | |
| | AGCACCTACGTGAAGAATGGTACCTCGTTTGA | |
| | CATCCACTATGGCTCGGGCAGCCTCTCCGGGT | |
| | ACCTGAGCCAGGACACTGTGTCGGTGCCCTGC | |
| | CAGTCAGCGTCGTCAGCCTCTGCCCTGGGCGG | |
| | TGTCAAAGTGGAGAGGCAGGTCTTTGGGGAGG | |
| | CCACCAAGCAGCCAGGCATCACCTTCATCGCA | |
| | GCCAAGTTCGATGGCATCCTGGGCATGGCCTA | |
| | CCCCCGCATCTCCGTCAACAACGTGCTGCCCG | |
| | TCTTCGACAACCTGATGCAGCAGAAGCTGGTG | |
| | GACCAGAACATCTTCTCCTTCTACCTGAGCAG | |
| | GGACCCAGATGCGCAGCCTGGGGGTGAGCTGA | |
| | TGCTGGGTGGCACAGACTCCAAGTATTACAAG | |
| | GGTTCTCTGTCCTACCTGAATGTCACCCGCAA | |
| | GGCCTACTGGCAGGTCCACCTGGACCAGGTGG | |
| | AGGTGGCCAGCGGGCTGACCCTGTGCAAGGAG | |
| | GGCTGTGAGGCCATTGTGGACACAGGCACTTC | |
| | CCTCATGGTGGGCCCGGTGGATGAGGTGCGCG | |
| | AGCTGCAGAAGGCCATCGGGGCCGTGCCGCTG | |
| | ATTCAGGGCGAGTACATGATCCCCTGTGAGAA | |
| | GGTGTCCACCCTGCCCGCGATCACACTGAAGC | |
| | TGGGAGGCAAAGGCTACAAGCTGTCCCCAGAG | |
| | GACTACACGCTCAAGGTGTCGCAGGCCGGGAA | |
| | GACCCTCTGCCTGAGCGGCTTCATGGGCATGG | |
| | ACATCCCGCCACCCAGCGGGCCACTCTGGATC | |
| | CTGGGCGACGTCTTCATCGGCCGCTACTACAC | |
| | TGTGTTTGACCGTGACAACAACAGGGTGGGCT | |
| | TCGCCGAGGCTGCCCGCCTCTAG | |
| Human | MQPSSLLPLALCLLAAPASALVRIPLHKFTSI | SEQ ID NO. 54 |
| cathepsin D | RRTMSEVGGSVEDLIAKGPVSKYSQAVPAVTE | |
| (CTSD) amino | GPIPEVLKNYMDAQYYGEIGIGTPPQCFTVVF | |
| acid | DTGSSNLWVPSIHCKLLDIACWIHHKYNSDKS | |
| sequence; | STYVKNGTSFDIHYGSGSLSGYLSQDTVSVPC | |
| | QSASSASALGGVKVERQVFGEATKQPGITFIA | |
| | AKFDGILGMAYPRISVNNVLPVFDNLMQQKLV | |
| | DQNIFSFYLSRDPDAQPGGELMLGGTDSKYYK | |
| | GSLSYLNVTRKAYWQVHLDQVEVASGLTLCKE | |
| | GCEAIVDTGTSLMVGPVDEVRELQKAIGAVPL | |
| | IQGEYMIPCEKVSTLPAITLKLGGKGYKLSPE | |
| | DYTLKVSQAGKTLCLSGFMGMDIPPPSGPLWI | |
| | LGDVFIGRYYTVFDRDNNRVGFAEAARL | |
| Human | ATGGCGTCGCCCGGCTGCCTGTGGCTCTTGGC | SEQ ID NO. 55 |
| palmitoyl | TGTGGCTCTCCTGCCATGGACCTGCGCTTCTC | |
| protein | GGGCGCTGCAGCATCTGGACCCGCCGGCGCCG | |
| thioesterase | CTGCCGTTGGTGATCTGGCATGGGATGGGAGA | |
| 1 (PPT1) | CAGCTGTTGCAATCCCTTAAGCATGGGTGCTA | |
| cDNA; | TTAAAAAAATGGTGGAGAAGAAAATACCTGGA | |
| | ATTTACGTCTTATCTTTAGAGATTGGGAAGAC | |
| | CCTGATGGAGGACGTGGAGAACAGCTTCTTCT | |
| | TGAATGTCAATTCCCAAGTAACAACAGTGTGT | |
| | CAGGCACTTGCTAAGGATCCTAAATTGCAGCA | |
| | AGGCTACAATGCTATGGGATTCTCCCAGGGAG | |
| | GCCAATTTCTGAGGGCAGTGGCTCAGAGATGC | |
| | CCTTCACCTCCCATGATCAATCTGATCTCGGT | |
| | TGGGGGACAACATCAAGGTGTTTTTGGACTCC | |
| | CTCGATGCCCAGGAGAGAGCTCTCACATCTGT | |
| | GACTTCATCCGAAAAACACTGAATGCTGGGGC | |
| | GTACTCCAAAGTTGTTCAGGAACGCCTCGTGC | |
| | AAGCCGAATACTGGCATGACCCCATAAAGGAG | |
| | GATGTGTATCGCAACCACAGCATCTTCTTGGC | |
| | AGATATAAATCAGGAGCGGGGTATCAATGAGT | |
| | CCTACAAGAAAAACCTGATGGCCCTGAAGAAG | |
| | TTTGTGATGGTGAAATTCCTCAATGATTCCAT | |
| | TGTGGACCCTGTAGATTCGGAGTGGTTTGGAT | |
| | TTTACAGAAGTGGCCAAGCCAAGGAAACCATT | |
| | CCCTTACAGGAGACCTCCCTGTACACACAGGA | |
| | CCGCCTGGGGCTAAAGGAAATGGACAATGCAG | |
| | GACAGCTAGTGTTTCTGGCTACAGAAGGGGAC | |
| | CATCTTCAGTTGTCTGAAGAATGGTTTTATGC | |
| | CCACATCATACCATTCCTTGGATGA | |
| Human | MASPGCLWLLAVALLPWTCASRALQHLDPPAP | SEQ ID NO. 56 |
| palmitoyl | LPLVIWHGMGDSCCNPLSMGAIKKMVEKKIPG | |
| protein | IYVLSLEIGKTLMEDVENSFFLNVNSQVTTVC | |
| thioesterase | QALAKDPKLQQGYNAMGFSQGGQFLRAVAQRC | |
| 1 (PPT1) | PSPPMINLISVGGQHQGVFGLPRCPGESSHIC | |
| amino acid | DFIRKTLNAGAYSKVVQERLVQAEYWHDPIKE | |
| sequence; | DVYRNHSIFLADINQERGINESYKKNLMALKK | |
| | FVMVKFLNDSIVDPVDSEWFGFYRSGQAKETI | |
| | PLQETSLYTQDRLGLKEMDNAGQLVFLATEGD | |
| | HLQLSEEWFYAHIIPFLG | |
| Human | ATGAGCTGCCCCGTGCCCGCCTGCTGCGCGCT | SEQ ID NO. 57 |
| sulfamidase | GCTGCTAGTCCTGGGGCTCTGCCGGGCGCGTC | |
| (SGSH) cDNA; | CCCGGAACGCACTGCTGCTCCTCGCGGATGAC | |
| | GGAGGCTTTGAGAGTGGCGCGTACAACAACAG | |
| | CGCCATCGCCACCCCGCACCTGGACGCCTTGG | |
| | CCCGCCGCAGCCTCCTCTTTCGCAATGCCTTC | |
| | ACCTCGGTCAGCAGCTGCTCTCCCAGCCGCGC | |
| | CAGCCTCCTCACTGGCCTGCCCCAGCATCAGA | |
| | ATGGGATGTACGGGCTGCACCAGGACGTGCAC | |
| | CACTTCAACTCCTTCGACAAGGTGCGGAGCCT | |
| | GCCGCTGCTGCTCAGCCAAGCTGGTGTGCGCA | |
| | CAGGCATCATCGGGAAGAAGCACGTGGGGCCG | |
| | GAGACCGTGTACCCGTTTGACTTTGCGTACAC | |
| | GGAGGAGAATGGCTCCGTCCTCCAGGTGGGGC | |
| | GGAACATCACTAGAATTAAGCTGCTCGTCCGG | |
| | AAATTCCTGCAGACTCAGGATGACCGGCCTTT | |
| | CTTCCTCTACGTCGCCTTCCACGACCCCCACC | |
| | GCTGTGGGCACTCCCAGCCCCAGTACGGAACC | |
| | TTCTGTGAGAAGTTTGGCAACGGAGAGAGCGG | |
| | CATGGGTCGTATCCCAGACTGGACCCCCCAGG | |
| | CCTACGACCCACTGGACGTGCTGGTGCCTTAC | |
| | TTCGTCCCCAACACCCCGGCAGCCCGAGCCGA | |
| | CCTGGCCGCTCAGTACACCACCGTAGGCCGCA | |
| | TGGACCAAGGAGTTGGACTGGTGCTCCAGGAG | |
| | CTGCGTGACGCCGGTGTCCTGAACGACACACT | |
| | GGTGATCTTCACGTCCGACAACGGGATCCCCT | |
| | TCCCCAGCGGCAGGACCAACCTGTACTGGCCG | |
| | GGCACTGCTGAACCCTTACTGGTGTCATCCCC | |
| | GGAGCACCCAAAACGCTGGGGCCAAGTCAGCG | |
| | AGGCCTACGTGAGCCTCCTAGACCTCACGCCC | |
| | ACCATCTTGGATTGGTTCTCGATCCCGTACCC | |
| | CAGCTACGCCATCTTTGGCTCGAAGACCATCC | |
| | ACCTCACTGGCCGGTCCCTCCTGCCGGCGCTG | |
| | GAGGCCGAGCCCCTCTGGGCCACCGTCTTTGG | |
| | CAGCCAGAGCCACCACGAGGTCACCATGTCCT | |
| | ACCCCATGCGCTCCGTGCAGCACCGGCACTTC | |
| | CGCCTCGTGCACAACCTCAACTTCAAGATGCC | |
| | CTTTCCCATCGACCAGGACTTCTACGTCTCAC | |
| | CCACCTTCCAGGACCTCCTGAACCGCACTACA | |
| | GCTGGTCAGCCCACGGGCTGGTACAAGGACCT | |
| | CCGTCATTACTACTACCGGGCGCGCTGGGAGC | |
| | TCTACGACCGGAGCCGGGACCCCCACGAGACC | |
| | CAGAACCTGGCCACCGACCCGCGCTTTGCTCA | |
| | GCTTCTGGAGATGCTTCGGGACCAGCTGGCCA | |
| | AGTGGCAGTGGGAGACCCACGACCCCTGGGTG | |
| | TGCGCCCCCGACGGCGTCCTGGAGGAGAAGCT | |
| | CTCTCCCCAGTGCCAGCCCCTCCACAATGAGC | |
| | TGTGA | |
| Human | MSCPVPACCALLLVLGLCRARPRNALLLLADD | SEQ ID NO. 58 |
| sulfamidase | GGFESGAYNNSAIATPHLDALARRSLLFRNAF | |
| (SGSH) amino | TSVSSCSPSRASLLTGLPQHQNGMYGLHQDVH | |
| acid | HFNSFDKVRSLPLLLSQAGVRTGIIGKKHVGP | |
| sequence; | ETVYPFDFAYTEENGSVLQVGRNITRIKLLVR | |
| | KFLQTQDDRPFFLYVAFHDPHRCGHSQPQYGT | |
| | FCEKFGNGESGMGRIPDWTPQAYDPLDVLVPY | |
| | FVPNTPAARADLAAQYTTVGRMDQGVGLVLQE | |
| | LRDAGVLNDTLVIFTSDNGIPFPSGRTNLYWP | |
| | GTAEPLLVSSPEHPKRWGQVSEAYVSLLDLTP | |
| | TILDWFSIPYPSYAIFGSKTIHLTGRSLLPAL | |
| | EAEPLWATVFGSQSHHEVTMSYPMRSVQHRHF | |
| | RLVHNLNFKMPFPIDQDFYVSPTFQDLLNRTT | |
| | AGQPTGWYKDLRHYYYRARWELYDRSRDPHET | |
| | QNLATDPRFAQLLEMLRDQLAKWQWETHDPWV | |
| | CAPDGVLEEKLSPQCQPLHNEL | |
| Human alpha- | ATGCGTCCCCTGCGCCCCCGCGCCGCGCTGCT | SEQ ID NO. 59 |
| L- | GGCGCTCCTGGCCTCGCTCCTGGCCGCGCCCC | |
| iduronidase | CGGTGGCCCCGGCCGAGGCCCCGCACCTGGTG | |
| (IDUA)cDNA; | CATGTGGACGCGGCCCGCGCGCTGTGGCCCCT | |
| | GCGGCGCTTCTGGAGGAGCACAGGCTTCTGCC | |
| | CCCCGCTGCCACACAGCCAGGCTGACCAGTAC | |
| | GTCCTCAGCTGGGACCAGCAGCTCAACCTCGC | |
| | CTATGTGGGCGCCGTCCCTCACCGCGGCATCA | |
| | AGCAGGTCCGGACCCACTGGCTGCTGGAGCTT | |
| | GTCACCACCAGGGGGTCCACTGGACGGGGCCT | |
| | GAGCTACAACTTCACCCACCTGGACGGGTACC | |
| | TGGACCTTCTCAGGGAGAACCAGCTCCTCCCA | |
| | GGGTTTGAGCTGATGGGCAGCGCCTCGGGCCA | |
| | CTTCACTGACTTTGAGGACAAGCAGCAGGTGT | |
| | TTGAGTGGAAGGACTTGGTCTCCAGCCTGGCC | |
| | AGGAGATACATCGGTAGGTACGGACTGGCGCA | |
| | TGTTTCCAAGTGGAACTTCGAGACGTGGAATG | |
| | AGCCAGACCACCACGACTTTGACAACGTCTCC | |
| | ATGACCATGCAAGGCTTCCTGAACTACTACGA | |
| | TGCCTGCTCGGAGGGTCTGCGCGCCGCCAGCC | |
| | CCGCCCTGCGGCTGGGAGGCCCCGGCGACTCC | |
| | TTCCACACCCCACCGCGATCCCCGCTGAGCTG | |
| | GGGCCTCCTGCGCCACTGCCACGACGGTACCA | |
| | ACTTCTTCACTGGGGAGGCGGGCGTGCGGCTG | |
| | GACTACATCTCCCTCCACAGGAAGGGTGCGCG | |
| | CAGCTCCATCTCCATCCTGGAGCAGGAGAAGG | |
| | TCGTCGCGCAGCAGATCCGGCAGCTCTTCCCC | |
| | AAGTTCGCGGACACCCCCATTTACAACGACGA | |
| | GGCGGACCCGCTGGTGGGCTGGTCCCTGCCAC | |
| | AGCCGTGGAGGGCGGACGTGACCTACGCGGCC | |
| | ATGGTGGTGAAGGTCATCGCGCAGCATCAGAA | |
| | CCTGCTACTGGCCAACACCACCTCCGCCTTCC | |
| | CCTACGCGCTCCTGAGCAACGACAATGCCTTC | |
| | CTGAGCTACCACCCGCACCCCTTCGCGCAGCG | |
| | CACGCTCACCGCGCGCTTCCAGGTCAACAACA | |
| | CCCGCCCGCCGCACGTGCAGCTGTTGCGCAAG | |
| | CCGGTGCTCACGGCCATGGGGCTGCTGGCGCT | |
| | GCTGGATGAGGAGCAGCTCTGGGCCGAAGTGT | |
| | CGCAGGCCGGGACCGTCCTGGACAGCAACCAC | |
| | ACGGTGGGCGTCCTGGCCAGCGCCCACCGCCC | |
| | CCAGGGCCCGGCCGACGCCTGGCGCGCCGCGG | |
| | TGCTGATCTACGCGAGCGACGACACCCGCGCC | |
| | CACCCCAACCGCAGCGTCGCGGTGACCCTGCG | |
| | GCTGCGCGGGGTGCCCCCCGGCCCGGGCCTGG | |
| | TCTACGTCACGCGCTACCTGGACAACGGGCTC | |
| | TGCAGCCCCGACGGCGAGTGGCGGCGCCTGGG | |
| | CCGGCCCGTCTTCCCCACGGCAGAGCAGTTCC | |
| | GGCGCATGCGCGCGGCTGAGGACCCGGTGGCC | |
| | GCGGCGCCCCGCCCCTTACCCGCCGGCGGCCG | |
| | CCTGACCCTGCGCCCCGCGCTGCGGCTGCCGT | |
| | CGCTTTTGCTGGTGCACGTGTGTGCGCGCCCC | |
| | GAGAAGCCGCCCGGGCAGGTCACGCGGCTCCG | |
| | CGCCCTGCCCCTGACCCAAGGGCAGCTGGTTC | |
| | TGGTCTGGTCGGATGAACACGTGGGCTCCAAG | |
| | TGCCTGTGGACATACGAGATCCAGTTCTCTCA | |
| | GGACGGTAAGGCGTACACCCCGGTCAGCAGGA | |
| | AGCCATCGACCTTCAACCTCTTTGTGTTCAGC | |
| | CCAGACACAGGTGCTGTCTCTGGCTCCTACCG | |
| | AGTTCGAGCCCTGGACTACTGGGCCCGACCAG | |
| | GCCCCTTCTCGGACCCTGTGCCGTACCTGGAG | |
| | GTCCCTGTGCCAAGAGGGCCCCCATCCCCGGG | |
| | CAATCCATGA | |
| Human alpha- | MRPLRPRAALLALLASLLAAPPVAPAEAPHLV | SEQ ID NO. 60 |
| L- | HVDAARALWPLRRFWRSTGFCPPLPHSQADQY | |
| iduronidase | VLSWDQQLNLAYVGAVPHRGIKQVRTHWLLEL | |
| (IDUA) amino | VTTRGSTGRGLSYNFTHLDGYLDLLRENQLLP | |
| acid | GFELMGSASGHFTDFEDKQQVFEWKDLVSSLA | |
| sequence; | RRYIGRYGLAHVSKWNFETWNEPDHHDFDNVS | |
| | MTMQGFLNYYDACSEGLRAASPALRLGGPGDS | |
| | FHTPPRSPLSWGLLRHCHDGTNFFTGEAGVRL | |
| | DYISLHRKGARSSISILEQEKVVAQQIRQLFP | |
| | KFADTPIYNDEADPLVGWSLPQPWRADVTYAA | |
| | MVVKVIAQHQNLLLANTTSAFPYALLSNDNAF | |
| | LSYHPHPFAQRTLTARFQVNNTRPPHVQLLRK | |
| | PVLTAMGLLALLDEEQLWAEVSQAGTVLDSNH | |
| | TVGVLASAHRPQGPADAWRAAVLIYASDDTRA | |
| | HPNRSVAVTLRLRGVPPGPGLVYVTRYLDNGL | |
| | CSPDGEWRRLGRPVFPTAEQFRRMRAAEDPVA | |
| | AAPRPLPAGGRLTLRPALRLPSLLLVHVCARP | |
| | EKPPGQVTRLRALPLTQGQLVLVWSDEHVGSK | |
| | CLWTYEIQFSQDGKAYTPVSRKPSTFNLFVFS | |
| | PDTGAVSGSYRVRALDYWARPGPFSDPVPYLE | |
| | VPVPRGPPSPGNP | |
| Human | ATGCCGCCACCCCGGACCGGCCGAGGCCTTCT | SEQ ID NO. 61 |
| iduronate-2- | CTGGCTGGGTCTGGTTCTGAGCTCCGTCTGCG | |
| sulfatase | TCGCCCTCGGATCCGAAACGCAGGCCAACTCG | |
| (IDS) cDNA; | ACCACAGATGCTCTGAACGTTCTTCTCATCAT | |
| | CGTGGATGACCTGCGCCCCTCCCTGGGCTGTT | |
| | ATGGGGATAAGCTGGTGAGGTCCCCAAATATT | |
| | GACCAACTGGCATCCCACAGCCTCCTCTTCCA | |
| | GAATGCCTTTGCGCAGCAAGCAGTGTGCGCCC | |
| | CGAGCCGCGTTTCTTTCCTCACTGGCAGGAGA | |
| | CCTGACACCACCCGCCTGTACGACTTCAACTC | |
| | CTACTGGAGGGTGCACGCTGGAAACTTCTCCA | |
| | CCATCCCCCAGTACTTCAAGGAGAATGGCTAT | |
| | GTGACCATGTCGGTGGGAAAAGTCTTTCACCC | |
| | TGGGATATCTTCTAACCATACCGATGATTCTC | |
| | CGTATAGCTGGTCTTTTCCACCTTATCATCCT | |
| | TCCTCTGAGAAGTATGAAAACACTAAGACATG | |
| | TCGAGGGCCAGATGGAGAACTCCATGCCAACC | |
| | TGCTTTGCCCTGTGGATGTGCTGGATGTTCCC | |
| | GAGGGCACCTTGCCTGACAAACAGAGCACTGA | |
| | GCAAGCCATACAGTTGTTGGAAAAGATGAAAA | |
| | CGTCAGCCAGTCCTTTCTTCCTGGCCGTTGGG | |
| | TATCATAAGCCACACATCCCCTTCAGATACCC | |
| | CAAGGAATTTCAGAAGTTGTATCCCTTGGAGA | |
| | ACATCACCCTGGCCCCCGATCCCGAGGTCCCT | |
| | GATGGCCTACCCCCTGTGGCCTACAACCCCTG | |
| | GATGGACATCAGGCAACGGGAAGACGTCCAAG | |
| | CCTTAAACATCAGTGTGCCGTATGGTCCAATT | |
| | CCTGTGGACTTTCAGCGGAAAATCCGCCAGAG | |
| | CTACTTTGCCTCTGTGTCATATTTGGATACAC | |
| | AGGTCGGCCGCCTCTTGAGTGCTTTGGACGAT | |
| | CTTCAGCTGGCCAACAGCACCATCATTGCATT | |
| | TACCTCGGATCATGGGTGGGCTCTAGGTGAAC | |
| | ATGGAGAATGGGCCAAATACAGCAATTTTGAT | |
| | GTTGCTACCCATGTTCCCCTGATATTCTATGT | |
| | TCCTGGAAGGACGGCTTCACTTCCGGAGGCAG | |
| | GCGAGAAGCTTTTCCCTTACCTCGACCCTTTT | |
| | GATTCCGCCTCACAGTTGATGGAGCCAGGCAG | |
| | GCAATCCATGGACCTTGTGGAACTTGTGTCTC | |
| | TTTTTCCCACGCTGGCTGGACTTGCAGGACTG | |
| | CAGGTTCCACCTCGCTGCCCCGTTCCTTCATT | |
| | TCACGTTGAGCTGTGCAGAGAAGGCAAGAACC | |
| | TTCTGAAGCATTTTCGATTCCGTGACTTGGAA | |
| | GAGGATCCGTACCTCCCTGGTAATCCCCGTGA | |
| | ACTGATTGCCTATAGCCAGTATCCCCGGCCTT | |
| | CAGACATCCCTCAGTGGAATTCTGACAAGCCG | |
| | AGTTTAAAAGATATAAAGATCATGGGCTATTC | |
| | CATACGCACCATAGACTATAGGTATACTGTGT | |
| | GGGTTGGCTTCAATCCTGATGAATTTCTAGCT | |
| | AACTTTTCTGACATCCATGCAGGGGAACTGTA | |
| | TTTTGTGGATTCTGACCCATTGCAGGATCACA | |
| | ATATGTATAATGATTCCCAAGGTGGAGATCTT | |
| | TTCCAGTTGTTGATGCCTTGA | |
| Human | MPPPRTGRGLLWLGLVLSSVCVALGSETQANS | SEQ ID NO. 62 |
| iduronate-2- | TTDALNVLLIIVDDLRPSLGCYGDKLVRSPNI | |
| sulfatase | DQLASHSLLFQNAFAQQAVCAPSRVSFLTGRR | |
| (IDS) amino | PDTTRLYDFNSYWRVHAGNFSTIPQYFKENGY | |
| acid | VTMSVGKVFHPGISSNHTDDSPYSWSFPPYHP | |
| sequence; | SSEKYENTKTCRGPDGELHANLLCPVDVLDVP | |
| | EGTLPDKQSTEQAIQLLEKMKTSASPFFLAVG | |
| | YHKPHIPFRYPKEFQKLYPLENITLAPDPEVP | |
| | DGLPPVAYNPWMDIRQREDVQALNISVPYGPI | |
| | PVDFQRKIRQSYFASVSYLDTQVGRLLSALDD | |
| | LQLANSTIIAFTSDHGWALGEHGEWAKYSNFD | |
| | VATHVPLIFYVPGRTASLPEAGEKLFPYLDPF | |
| | DSASQLMEPGRQSMDLVELVSLFPTLAGLAGL | |
| | QVPPRCPVPSFHVELCREGKNLLKHFRFRDLE | |
| | EDPYLPGNPRELIAYSQYPRPSDIPQWNSDKP | |
| | SLKDIKIMGYSIRTIDYRYTVWVGFNPDEFLA | |
| | NFSDIHAGELYFVDSDPLQDHNMYNDSQGGDL | |
| | FQLLMP | |
| Human | ATGGGGGCACCGCGGTCCCTCCTCCTGGCCCT | SEQ ID NO. 63 |
| arylsulfatase | GGCTGCTGGCCTGGCCGTTGCCCGTCCGCCCA | |
| A (ARSA) | ACATCGTGCTGATCTTTGCCGACGACCTCGGC | |
| cDNA. | TATGGGGACCTGGGCTGCTATGGGCACCCCAG | |
| Remark: for | CTCTACCACTCCCAACCTGGACCAGCTGGCGG | |
| the C- | CGGGAGGGCTGCGGTTCACAGACTTCTACGTG | |
| terminal | CCTGTGTCTCTGTGCACACCCTCTAGGGCCGC | |
| tags the | CCTCCTGACCGGCCGGCTCCCGGTTCGGATGG | |
| sequence | GCATGTACCCTGGCGTCCTGGTGCCCAGCTCC | |
| “CATGCC” | CGGGGGGGCCTGCCCCTGGAGGAGGTGACCGT | |
| immediately | GGCCGAAGTCCTGGCTGCCCGAGGCTACCTCA | |
| before the | CAGGAATGGCCGGCAAGTGGCACCTTGGGGTG | |
| stop codon | GGGCCTGAGGGGGCCTTCCTGCCCCCCCATCA | |
| was omitted | GGGCTTCCATCGATTTCTAGGCATCCCGTACT | |
| (small | CCCACGACCAGGGCCCCTGCCAGAACCTGACC | |
| letters); | TGCTTCCCGCCGGCCACTCCTTGCGACGGTGG | |
| | CTGTGACCAGGGCCTGGTCCCCATCCCACTGT | |
| | TGGCCAACCTGTCCGTGGAGGCGCAGCCCCCC | |
| | TGGCTGCCCGGACTAGAGGCCCGCTACATGGC | |
| | TTTCGCCCATGACCTCATGGCCGACGCCCAGC | |
| | GCCAGGATCGCCCCTTCTTCCTGTACTATGCC | |
| | TCTCACCACACCCACTACCCTCAGTTCAGTGG | |
| | GCAGAGCTTTGCAGAGCGTTCAGGCCGCGGGC | |
| | CATTTGGGGACTCCCTGATGGAGCTGGATGCA | |
| | GCTGTGGGGACCCTGATGACAGCCATAGGGGA | |
| | CCTGGGGCTGCTTGAAGAGACGCTGGTCATCT | |
| | TCACTGCAGACAATGGACCTGAGACCATGCGT | |
| | ATGTCCCGAGGCGGCTGCTCCGGTCTCTTGCG | |
| | GTGTGGAAAGGGAACGACCTACGAGGGCGGTG | |
| | TCCGAGAGCCTGCCTTGGCCTTCTGGCCAGGT | |
| | CATATCGCTCCCGGCGTGACCCACGAGCTGGC | |
| | CAGCTCCCTGGACCTGCTGCCTACCCTGGCAG | |
| | CCCTGGCTGGGGCCCCACTGCCCAATGTCACC | |
| | TTGGATGGCTTTGACCTCAGCCCCCTGCTGCT | |
| | GGGCACAGGCAAGAGCCCTCGGCAGTCTCTCT | |
| | TCTTCTACCCGTCCTACCCAGACGAGGTCCGT | |
| | GGGGTTTTTGCTGTGCGGACTGGAAAGTACAA | |
| | GGCTCACTTCTTCACCCAGGGCTCTGCCCACA | |
| | GTGATACCACTGCAGACCCTGCCTGCCACGCC | |
| | TCCAGCTCTCTGACTGCTCATGAGCCCCCGCT | |
| | GCTCTATGACCTGTCCAAGGACCCTGGTGAGA | |
| | ACTACAACCTGCTGGGGGGTGTGGCCGGGGCC | |
| | ACCCCAGAGGTGCTGCAAGCCCTGAAACAGCT | |
| | TCAGCTGCTCAAGGCCCAGTTAGACGCAGCTG | |
| | TGACCTTCGGCCCCAGCCAGGTGGCCCGGGGC | |
| | GAGGACCCCGCCCTGCAGATCTGCTGTCATCC | |
| | TGGCTGCACCCCCCGCCCAGCTTGCTGCCATT | |
| | GCCCAGATCCCcatgccTGA | |
| Human | MGAPRSLLLALAAGLAVARPPNIVLIFADDLG | SEQ ID NO. 64 |
| arylsulfatase | YGDLGCYGHPSSTTPNLDQLAAGGLRFTDFYV | |
| A (ARSA) | PVSLCTPSRAALLTGRLPVRMGMYPGVLVPSS | |
| amino acid | RGGLPLEEVTVAEVLAARGYLTGMAGKWHLGV | |
| sequence. | GPEGAFLPPHQGFHRFLGIPYSHDQGPCQNLT | |
| Remark: for | CFPPATPCDGGCDQGLVPIPLLANLSVEAQPP | |
| the C- | WLPGLEARYMAFAHDLMADAQRQDRPFFLYYA | |
| terminal | SHHTHYPQFSGQSFAERSGRGPFGDSLMELDA | |
| tags the | AVGTLMTAIGDLGLLEETLVIFTADNGPETMR | |
| last two | MSRGGCSGLLRCGKGTTYEGGVREPALAFWPG | |
| amino acids | HIAPGVTHELASSLDLLPTLAALAGAPLPNVT | |
| “HA” were | LDGFDLSPLLLGTGKSPRQSLFFYPSYPDEVR | |
| omitted; | GVFAVRTGKYKAHFFTQGSAHSDTTADPACHA | |
| | SSSLTAHEPPLLYDLSKDPGENYNLLGGVAGA | |
| | TPEVLQALKQLQLLKAQLDAAVTFGPSQVARG | |
| | EDPALQICCHPGCTPRPACCHCPDPHA | |
| Human | ATGGCTGCAGCCGCGGGTTCGGCGGGCCGCGC | SEQ ID NO. 65 |
| galacto- | CGCGGTGCCCTTGCTGCTGTGTGCGCTGCTGG | |
| cerebrosidase | CGCCCGGCGGCGCGTACGTGCTCGACGACTCC | |
| (GALC) cDNA. | GACGGGCTGGGCCGGGAGTTCGACGGCATCGG | |
| Remark: for | CGCGGTCAGCGGCGGCGGGGCAACCTCCCGAC | |
| the C- | TTCTAGTAAATTACCCAGAGCCCTATCGTTCT | |
| terminal | CAGATATTGGATTATCTCTTTAAGCCGAATTT | |
| tags the | TGGTGCCTCTTTGCATATTTTAAAAGTGGAAA | |
| sequence | TAGGTGGTGATGGGCAGACAACAGATGGCACT | |
| “CGC” | GAGCCCTCCCACATGCATTATGCACTAGATGA | |
| immediately | GAATTATTTCCGAGGATACGAGTGGTGGTTGA | |
| before the | TGAAAGAAGCTAAGAAGAGGAATCCCAATATT | |
| stop codon | ACACTCATTGGGTTGCCATGGTCATTCCCTGG | |
| was omitted | ATGGCTGGGAAAAGGTTTCGACTGGCCTTATG | |
| (small | TCAATCTTCAGCTGACTGCCTATTATGTCGTG | |
| letters); | ACCTGGATTGTGGGCGCCAAGCGTTACCATGA | |
| | TTTGGACATTGATTATATTGGAATTTGGAATG | |
| | AGAGGTCATATAATGCCAATTATATTAAGATA | |
| | TTAAGAAAAATGCTGAATTATCAAGGTCTCCA | |
| | GCGAGTGAAAATCATAGCAAGTGATAATCTCT | |
| | GGGAGTCCATCTCTGCATCCATGCTCCTTGAT | |
| | GCCGAACTCTTCAAGGTGGTTGATGTTATAGG | |
| | GGCTCATTATCCTGGAACCCATTCAGCAAAAG | |
| | ATGCAAAGTTGACTGGGAAGAAGCTTTGGTCT | |
| | TCTGAAGACTTTAGCACTTTAAATAGTGACAT | |
| | GGGTGCAGGCTGCTGGGGTCGCATTTTAAATC | |
| | AGAATTATATCAATGGCTATATGACTTCCACA | |
| | ATCGCATGGAATTTAGTGGCTAGTTACTATGA | |
| | ACAGTTGCCTTATGGGAGATGCGGGTTGATGA | |
| | CGGCCCAGGAGCCATGGAGTGGGCACTACGTG | |
| | GTAGAATCTCCTGTCTGGGTATCAGCTCATAC | |
| | CACTCAGTTTACTCAACCTGGCTGGTATTACC | |
| | TGAAGACAGTTGGCCATTTAGAGAAAGGAGGA | |
| | AGCTACGTAGCTCTGACTGATGGCTTAGGGAA | |
| | CCTCACCATCATCATTGAAACCATGAGTCATA | |
| | AACATTCTAAGTGCATACGGCCATTTCTTCCT | |
| | TATTTCAATGTGTCACAACAATTTGCCACCTT | |
| | TGTTCTTAAGGGATCTTTTAGTGAAATACCAG | |
| | AGCTACAGGTATGGTATACCAAACTTGGAAAA | |
| | ACATCCGAAAGATTTCTTTTTAAGCAGCTGGA | |
| | TTCTCTATGGCTCCTTGACAGCGATGGCAGTT | |
| | TCACACTGAGCCTGCATGAAGATGAGCTGTTC | |
| | ACACTCACCACTCTCACCACTGGTCGCAAAGG | |
| | CAGCTACCCGCTTCCTCCAAAATCCCAGCCCT | |
| | TCCCAAGTACCTATAAGGATGATTTCAATGTT | |
| | GATTACCCATTTTTTAGTGAAGCTCCAAACTT | |
| | TGCTGATCAAACTGGTGTATTTGAATATTTTA | |
| | CAAATATTGAAGACCCTGGCGAGCATCACTTC | |
| | ACGCTACGCCAAGTTCTCAACCAGAGACCCAT | |
| | TACGTGGGCTGCCGATGCATCCAACACAATCA | |
| | GTATTATAGGAGACTACAACTGGACCAATCTG | |
| | ACTACAAAGTGTGATGTTTACATAGAGACCCC | |
| | TGACACAGGAGGTGTGTTCATTGCAGGAAGAG | |
| | TAAATAAAGGTGGTATTTTGATTAGAAGTGCC | |
| | AGAGGAATTTTCTTCTGGATTTTTGCAAATGG | |
| | ATCTTACAGGGTTACAGGTGATTTAGCTGGAT | |
| | GGATTATATATGCTTTAGGACGTGTTGAAGTT | |
| | ACAGCAAAAAAATGGTATACACTCACGTTAAC | |
| | TATTAAGGGTCATTTCGCCTCTGGCATGCTGA | |
| | ATGACAAGTCTCTGTGGACAGACATCCCTGTG | |
| | AATTTTCCAAAGAATGGCTGGGCTGCAATTGG | |
| | AACTCACTCCTTTGAATTTGCACAGTTTGACA | |
| | ACTTTCTTGTGGAAGCCACAcgcTAA | |
| Human | MAAAAGSAGRAAVPLLLCALLAPGGAYVLDDS | SEQ ID NO. 66 |
| galacto- | DGLGREFDGIGAVSGGGATSRLLVNYPEPYRS | |
| cerebrosidase | QILDYLFKPNFGASLHILKVEIGGDGQTTDGT | |
| (GALC) amino | EPSHMHYALDENYFRGYEWWLMKEAKKRNPNI | |
| acid | TLIGLPWSFPGWLGKGFDWPYVNLQLTAYYVV | |
| sequence. | TWIVGAKRYHDLDIDYIGIWNERSYNANYIKI | |
| Remark: for | LRKMLNYQGLQRVKIIASDNLWESISASMLLD | |
| the C- | AELFKVVDVIGAHYPGTHSAKDAKLTGKKLWS | |
| terminal | SEDFSTLNSDMGAGCWGRILNQNYINGYMTST | |
| tags the | IAWNLVASYYEQLPYGRCGLMTAQEPWSGHYV | |
| last amino | VESPVWVSAHTTQFTQPGWYYLKTVGHLEKGG | |
| acid “R” was | SYVALTDGLGNLTIIIETMSHKHSKCIRPFLP | |
| omitted; | YFNVSQQFATFVLKGSFSEIPELQVWYTKLGK | |
| | TSERFLFKQLDSLWLLDSDGSFTLSLHEDELF | |
| | TLTTLTTGRKGSYPLPPKSQPFPSTYKDDFNV | |
| | DYPFFSEAPNFADQTGVFEYFTNIEDPGEHHF | |
| | TLRQVLNQRPITWAADASNTISIIGDYNWTNL | |
| | TTKCDVYIETPDTGGVFIAGRVNKGGILIRSA | |
| | RGIFFWIFANGSYRVTGDLAGWIIYALGRVEV | |
| | TAKKWYTLTLTIKGHFASGMLNDKSLWTDIPV | |
| | NFPKNGWAAIGTHSFEFAQFDNFLVEATR | |
| Human acid | ATGGAGTTTTCAAGTCCTTCCAGAGAGGAATG | SEQ ID NO. 67 |
| beta- | TCCCAAGCCTTTGAGTAGGGTAAGCATCATGG | |
| glucosidase = | CTGGCAGCCTCACAGGATTGCTTCTACTTCAG | |
| beta- | GCAGTGTCGTGGGCATCAGGTGCCCGCCCCTG | |
| glucocere- | CATCCCTAAAAGCTTCGGCTACAGCTCGGTGG | |
| brosidase | TGTGTGTCTGCAATGCCACATACTGTGACTCC | |
| (GBA) cDNA. | TTTGACCCCCCGACCTTTCCTGCCCTTGGTAC | |
| Remark: | CTTCAGCCGCTATGAGAGTACACGCAGTGGGC | |
| substitution | GACGGATGGAGCTGAGTATGGGGCCCATCCAG | |
| of 3 | GCTAATCACACGGGCACAGGCCTGCTACTGAC | |
| cysteine by | CCTGCAGCCAGAACAGAAGTTCCAGAAAGTGA | |
| serine | AGGGATTTGGAGGGGCCATGACAGATGCTGCT | |
| residues; | GCTCTCAACATCCTTGCCCTGTCACCCCCTGC | |
| | CCAAAATTTGCTACTTAAATCGTACTTCTCTG | |
| | AAGAAGGAATCGGATATAACATCATCCGGGTA | |
| | CCCATGGCCAGCAGCGACTTCTCCATCCGCAC | |
| | CTACACCTATGCAGACACCCCTGATGATTTCC | |
| | AGTTGCACAACTTCAGCCTCCCAGAGGAAGAT | |
| | ACCAAGCTCAAGATACCCCTGATTCACCGAGC | |
| | CCTGCAGTTGGCCCAGCGTCCCGTTTCACTCC | |
| | TTGCCAGCCCCTGGACATCACCCACTTGGCTC | |
| | AAGACCAATGGAGCGGTGAATGGGAAGGGGTC | |
| | ACTCAAGGGACAGCCCGGAGACATCTACCACC | |
| | AGACCTGGGCCAGATACTTTGTGAAGTTCCTG | |
| | GATGCCTATGCTGAGCACAAGTTACAGTTCTG | |
| | GGCAGTGACAGCTGAAAATGAGCCTTCTGCTG | |
| | GGCTGTTGAGTGGATACCCCTTCCAGAGCCTG | |
| | GGCTTCACCCCTGAACATCAGCGAGACTTCAT | |
| | TGCCCGTGACCTAGGTCCTACCCTCGCCAACA | |
| | GTACTCACCACAATGTCCGCCTACTCATGCTG | |
| | GATGACCAACGCTTGCTGCTGCCCCACTGGGC | |
| | AAAGGTGGTACTGACAGACCCAGAAGCAGCTA | |
| | AATATGTTCATGGCATTGCTGTACATTGGTAC | |
| | CTGGACTTTCTGGCTCCAGCCAAAGCCACCCT | |
| | AGGGGAGACACACCGCCTGTTCCCCAACACCA | |
| | TGCTCTTTGCCTCAGAGGCCAGCGTGGGCTCC | |
| | AAGTTCTGGGAGCAGAGTGTGCGGCTAGGCTC | |
| | CTGGGATCGAGGGATGCAGTACAGCCACAGCA | |
| | TCATCACGAACCTCCTGTACCATGTGGTCGGC | |
| | TGGACCGACTGGAACCTTGCCCTGAACCCCGA | |
| | AGGAGGACCCAATTGGGTGCGTAACTTTGTCG | |
| | ACAGTCCCATCATTGTAGACATCACCAAGGAC | |
| | ACGTTTTACAAACAGCCCATGTTCTACCACCT | |
| | TGGCCACTTCAGCAAGTTCATTCCTGAGGGCT | |
| | CCCAGAGAGTGGGGCTGGTTGCCAGTCAGAAG | |
| | AACGACCTGGACGCAGTGGCACTGATGCATCC | |
| | CGATGGCTCTGCTGTTGTGGTCGTGCTAAACC | |
| | GCTCCTCTAAGGATGTGCCTCTTACCATCAAG | |
| | GATCCTGCTGTGGGCTTCCTGGAGACAATCTC | |
| | ACCTGGCTACTCCATTCACACCTACCTGTGGC | |
| | GTCGCCAGTGA | |
| Human acid | ASMEFSSPSREECPKPLSRVSIMAGSLTGLLL | SEQ ID NO. 68 |
| beta- | LQAVSWASGARPCIPKSFGYSSVVCVCNATYC | |
| glucosidase = | DSFDPPTFPALGTFSRYESTRSGRRMELSMGP | |
| beta- | IQANHTGTGLLLTLQPEQKFQKVKGFGGAMTD | |
| glucocere- | AAALNILALSPPAQNLLLKSYFSEEGIGYNII | |
| brosidase | RVPMASSDFSIRTYTYADTPDDFQLHNPSLPE | |
| (GBA) | EDTKLKIPLIHRALQLAQRPVSLLASPWTSPT | |
| amino acid | WLKTNGAVNGKGSLKGQPGDIYHQTWARYFVK | |
| sequence. | FLDAYAEHKLQFWAVTAENEPSAGLLSGYPFQ | |
| Remark: | SLGFTPEHQRDFIARDLGPTLANSTHHNVRLL | |
| substitution | MLDDQRLLLPHWAKVVLTDPEAAKYVHGIAVH | |
| of 3 “C” by | WYLDFLAPAKATLGETHRLFPNTMLFASEASV | |
| “S” (C165S, | GSKFWEQSVRLGSWDRGMQYSHSIITNLLYHV | |
| C287S, | VGWTDWNLALNPEGGPNWVRNFVDSPIIVDIT | |
| C381S); | KDTFYKQPMFYHLGHFSKFIPEGSQRVGLVAS | |
| | QKNDLDAVALMHPDGSAVVVVLNRSSKDVPLT | |
| | IKDPAVGFLETISPGYSIHTYLWRRQ | |
| Human alpha | ATGCAGCTGAGGAACCCAGAACTACATCTGGG | SEQ ID NO. 69 |
| galactosidase | CTGCGCGCTTGCGCTTCGCTTCCTGGCCCTCG | |
| (GLA) | TTTCCTGGGACATCCCTGGGGCTAGAGCACTG | |
| cDNA; | GACAATGGATTGGCAAGGACGCCTACCATGGG | |
| | CTGGCTGCACTGGGAGCGCTTCATGTGCAACC | |
| | TTGACTGCCAGGAAGAGCCAGATTCCTGCATC | |
| | AGTGAGAAGCTCTTCATGGAGATGGCAGAGCT | |
| | CATGGTCTCAGAAGGCTGGAAGGATGCAGGTT | |
| | ATGAGTACCTCTGCATTGATGACTGTTGGATG | |
| | GCTCCCCAAAGAGATTCAGAAGGCAGACTTCA | |
| | GGCAGACCCTCAGCGCTTTCCTCATGGGATTC | |
| | GCCAGCTAGCTAATTATGTTCACAGCAAAGGA | |
| | CTGAAGCTAGGGATTTATGCAGATGTTGGGAA | |
| | TAAAACCTGCGCAGGCTTCCCTGGGAGTTTTG | |
| | GATACTACGACATTGATGCCCAGACCTTTGCT | |
| | GACTGGGGAGTAGATCTGCTAAAATTTGATGG | |
| | TTGTTACTGTGACAGTTTGGAAAATTTGGCAG | |
| | ATGGTTATAAGCACATGTCCTTGGCCCTGAAT | |
| | AGGACTGGCAGAAGCATTGTGTACTCCTGTGA | |
| | GTGGCCTCTTTATATGTGGCCCTTTCAAAAGC | |
| | CCAATTATACAGAAATCCGACAGTACTGCAAT | |
| | CACTGGCGAAATTTTGCTGACATTGATGATTC | |
| | CTGGAAAAGTATAAAGAGTATCTTGGACTGGA | |
| | CATCTTTTAACCAGGAGAGAATTGTTGATGTT | |
| | GCTGGACCAGGGGGTTGGAATGACCCAGATAT | |
| | GTTAGTGATTGGCAACTTTGGCCTCAGCTGGA | |
| | ATCAGCAAGTAACTCAGATGGCCCTCTGGGCT | |
| | ATCATGGCTGCTCCTTTATTCATGTCTAATGA | |
| | CCTCCGACACATCAGCCCTCAAGCCAAAGCTC | |
| | TCCTTCAGGATAAGGACGTAATTGCCATCAAT | |
| | CAGGACCCCTTGGGCAAGCAAGGGTACCAGCT | |
| | TAGACAGGGAGACAACTTTGAAGTGTGGGAAC | |
| | GACCTCTCTCAGGCTTAGCCTGGGCTGTAGCT | |
| | ATGATAAACCGGCAGGAGATTGGTGGACCTCG | |
| | CTCTTATACCATCGCAGTTGCTTCCCTGGGTA | |
| | AAGGAGTGGCCTGTAATCCTGCCTGCTTCATC | |
| | ACACAGCTCCTCCCTGTGAAAAGGAAGCTAGG | |
| | GTTCTATGAATGGACTTCAAGGTTAAGAAGTC | |
| | ACATAAATCCCACAGGCACTGTTTTGCTTCAG | |
| | CTAGAAAATACAATGCAGATGTCATTAAAAGA | |
| | CTTACTTTAA | |
| Human alpha | MQLRNPELHLGCALALRFLALVSWDIPGARAL | SEQ ID NO. 70 |
| galactosidase | DNGLARTPTMGWLHWERFMCNLDCQEEPDSCI | |
| (GLA) | SEKLFMEMAELMVSEGWKDAGYEYLCIDDCWM | |
| amino acid | APQRDSEGRLQADPQRFPHGIRQLANYVHSKG | |
| sequence; | LKLGIYADVGNKTCAGFPGSFGYYDIDAQTFA | |
| | DWGVDLLKFDGCYCDSLENLADGYKHMSLALN | |
| | RTGRSIVYSCEWPLYMWPFQKPNYTEIRQYCN | |
| | HWRNFADIDDSWKSIKSILDWTSFNQERIVDV | |
| | AGPGGWNDPDMLVIGNFGLSWNQQVTQMALWA | |
| | IMAAPLFMSNDLRHISPQAKALLQDKDVIAIN | |
| | QDPLGKQGYQLRQGDNFEVWERPLSGLAWAVA | |
| | MINRQEIGGPRSYTIAVASLGKGVACNPACFI | |
| | TQLLPVKRKLGFYEWTSRLRSHINPTGTVLLQ | |
| | LENTMQMSLKDLL | |

In a preferred embodiment the targeting moiety is selected from the group of Antp, CLOCK, FGF2, HB1 and HB2 including the variants as outlined above.

Preferably the targeting moiety and the enzyme moiety are linked via a peptide linker as encoded by one of the following sequences SEQ ID NO. 15 or 16. SEQ ID NO. 21 or 22 are preferably N-terminal to FGF2 variants. SEQ ID NO. 43 or 44 are preferably N-terminal to the lysosomal proteins.

In one embodiment the peptide linker comprises a protease cleavage site. Such a site may be a site recognized by factor Xa, a caspase, thrombin, trypsin, papain and plasmin. For FGF2 variant constructs this is preferred.

In a preferred embodiment the lysosomal enzyme is selected from the group consisting of β-galactocerebrosidase, arylsulfatase A (sulfatidase), α-iduronidase, sulfarnimidase, α-N-acetylglucosaminidase, acetyl-CoA:α-glucosaminide-N-Ac-transferase, N-acetylglucosamine-6-sulfatase, tripeptidyl-peptidase 1, palmitoyl-protein thioesterase, β-galactosidase, sphingomyelinase, β-hexosaminidase A, β-hexosaminidase B, ceramidase, α-mannosidase, β-mannosidase, β-fucosidase, sialidase, α-N-acetylgalactosaminidase, α-L-iduronidase, iduronate-2-sulfatase, sulfamidase (heparan N-sulftase), α-N-acetylglucosaminidase, N-acetylgalactosamin-6-sulfatase, arylsulfatase B, β-glucuronidase, α-L-fucosidase, aspartylglucosaminidase, β-neuraminidase (sialidase), cathepsin A, β-hexosaminidase A+B, arylsulfatase A, cerebroside-β-galactosidase, β-glucocerebrosidase, β-galactosidase A (ceramide trihexosidase), acid α-glucosidase (acid maltase), CLN5-protein, acid lipase, steroid sulfatase (arylsulfatase C) and cathepsin D.

In a very preferred embodiment the lysosomal enzyme is selected from the group consisting of tripeptidyl-peptidase 1 (TPP1), Human cathepsin D (CTSD), Human palmitoyl protein thioesterase 1 (PPT1), Human sulfamidase (SGSH), Human alpha-L-iduronidase (IDUA), Human iduronate-2-sulfatase (IDS), Human arylsulfatase A (ARSA), Human acid beta-glucosidase-beta-glucocerebrosidase (GBA) and Human alpha-galactosidase (GLA).

Preferably the targeting moiety is a polypeptide having a sequence according to any one of SEQ ID NO. 18, 20, 24, 26, 27, 30, 71, 72 or as encoded by 23, 25, 27, 29, 43, and 44 or the other nucleic acid sequences encoding the respective peptides.

Polypeptides with reduced nuclear translocation are preferred, such as SEQ ID NO. 28.

Polypeptides with reduced FGF receptor binding are preferred, such as SEQ ID NO. 26.

Also polypeptides with both above mentioned activity reductions are preferred such as SEQ ID NO. 30.

Preferably the enzyme moiety is a polypeptide having a sequence according to any one of SEQ ID NO. 52, 54, 56, 58, 60, 62, 64, 66, 68 and 70.

Preferably chimeric molecule polypeptide has a sequence according to any one of the SEQ ID NO. 32, 34, 36, 38 40, 42, 46, 48 and 50.

The present invention also relates to sequence variants, allelic variants or mutants of the chimeric molecule described herein, and nucleic acid sequences that encode them. Sequence variants of the invention preferably share at least 90%, 91%, 92%, 93% or 94% identity with a polypeptide of the invention or with a nucleic acid sequence that encodes it. More preferably, a sequence variant shares at least 95%, 96%, 97% or 98% identity at the amino acid or nucleic acid level. Most preferably, a sequence variant shares at least 99%, 99.5%, 99.9% or more identity with a polypeptide of the invention or a nucleic acid sequence that encodes it.

Accordingly, the present invention provides an isolated chimeric protein comprising a sequence that is at least 80%, 85%, 90%, 92%, 94%, 95%, 96%, 97%, 98%, 99%, 99.5%, 99.9% or 100% identical to the sequences outlined above.

The chimeric molecules may be pegylated. The term “pegylation,” “polyethylene glycol” or “PEG” includes a polyalkylene glycol compound or a derivative thereof, with or without coupling agents or derivatization with coupling or activating moieties (e.g., with thiol, triflate, tresylate, azirdine, oxirane, or preferably with a maleimide moiety, e.g., PEG-maleimide). Other appropriate polyalkylene glycol compounds include, but are not limited to, maleimido monomethoxy PEG, activated PEG polypropylene glycol, but also charged or neutral polymers of the following types: dextran, colominic acids, or other carbohydrate based polymers, polymers of amino acids, and biotin and other affinity reagent derivatives.

The chimeric molecules may be incorporated into nanoparticles, solid polymeric molecules of 1-1000 nm diameters. These nanoparticles may comprise poly butyl cyanoacrylate, poly lactic acid or similar compounds and can be coated with polysorbate 80 and polysorbate 20 or similar non-ionic surfactant and emulsifier.

The chimeric molecules may be incorporated into virus like particles that consist of recombinantly produced viral envelope proteins. The chimeric molecules are packaged into these viral envelope proteins and taken up by cells via viral cell surface receptors and released from the viral envelope proteins within the target cells.

The invention also relates to a polynucleotide encoding the chimeric molecule according to the invention.

Preferred polynucleotides according to the invention are selected from the group of the SEQ ID NO. 31, 33, 35, 37, 39, 41, 45, 47 and 49.

The nucleic acid may differ from the sequence outlined above, in particular due to the degeneracy of the genetic code.

The invention also relates to a pharmaceutical composition comprising a chimeric molecule according to the invention.

The invention relates to the chimeric molecule according to the invention for the use in the treatment of a disease.

The disease is preferably a lysosomal storage disease, preferably with brain involvement.

Preferably the lysosomal storage disease is selected from the group consisting of the neuronal ceroid lipofuscinoses (NCL), infantile NCL (CLN1-defect), late infantile NCL (CLN2-defect), late infantile NCL (CLN5-defect), NCL caused by cathepsin D deficiency (CLN10-defect), mucopolysaccharidosis type I, mucopolysaccharidosis type II, mucopolysaccharidosis type IIIA, mucopolysaccharidosis type IIIB, mucopolysaccharidosis type WC, mucopolysaccharidosis type IIID, mucopolysaccharidosis type IVA, mucopolysaccharidosis type IVB, mucopolysaccharidosis type VI, mucopolysaccharidosis type VII, fucosidosis, α-mannosidosis, β-mannosidosis, aspartylglucosaminuria, Schindler's disease, sialidosis (mucolipidosis I), galactosialidosis, GM₁-gangliosidosis 1 mucopolysaccharidosis type IVB, GM₂-gangliosidosis, Sandhoff disease, Tay-Sachs disease, metachromatic leukodystrophy, Krabbe disease, Gaucher disease, Fabry disease, Niemann-Pick disease type A+B), glycogen storage disease type II (Pompe disease), Faber's syndrome, Wolman disease, X-linked ichthyosis.

Brain involvement in context of the present invention refers to diseases related to neurological and/or psychiatric symptoms, i.e. to any abnormality related to the central nervous system and may manifest as neurological or psychiatric symptoms (e.g. mental retardation), as neuropysiological abnormality (e.g. signs of epileptic discharges in the electroencephalography) or as abnormal brain imaging (e.g. atrophy of the grey matter).

In one embodiment of the present invention lysosomal storage diseases with brain involvement are selected from the group consisting of neuronal ceroid lipofuscinoses (NCL), infantile NCL (CLN1-defect), late infantile NCL (CLN2-defect), late infantile NCL (CLN5-defect), NCL caused by cathepsin D deficiency (CLN10-defect), mucopolysaccharidosis type I, mucopolysaccharidosis type II, mucopolysaccharidosis type IIIA, mucopolysaccharidosis type IIIB, mucopolysaccharidosis type IIIC, mucopolysaccharidosis type IIID, mucopolysaccharidosis type IVB, mucopolysaccharidosis type VII, fucosidosis, α-mannosidosis, β-mannosidosis, aspartylglucosaminuria, Schindler's disease, sialidosis (mucolipidosis I), galactosialidosis, GM₁-gangliosidosis/mucopolysaccharidosis type IVB, GM₂-gangliosidosis, Sandhoff disease, Tay-Sachs disease, metachromatic leukodystrophy, Krabbe disease, Gaucher disease, Fabry disease, Niemann-Pick disease (type A+B), Faber's syndrome, Wolman disease.

In one embodiment the lysosomal storage disease is the late infantile form of neuronal ceroid lipofuscinosis and the enzyme moiety comprises lysosomal tripeptidyl peptidase 1 (TPP1).

Combinations of disease names and enzyme defects are given in table 8 below.

TABLE 8

| DISEASE | ENZYME/PROTEIN DEFECT |
|---|---|
| Mucopolysaccharidosis type I | α-L-Iduronidase |
| Mucopolysaccharidosis type II | Iduronat-2-Sulfatase |
| Mucopolysaccharidosis type IIIA | Sulfamidase (Heparan N-Sulfatase) |
| Mucopolysaccharidosis type IIIB | α-N-Acetylglucosaminidase |
| Mucopolysaccharidosis type IIIC | Glucosamin-N-Acetyltransferase |
| Mucopolysaccharidosis type IIID | N-Acetylglucosamin-6-Sulfatase |
| Mucopolysaccharidosis type IVA | N-Acetylgalactosamin-6-Sulfatase |
| Mucopolysaccharidosis type IVB | β-Galactosidase |
| Mucopolysaccharidosis type VI | Arylsulfatase B |
| Mucopolysaccharidosis type VII | β-Glucuronidase |
| Fucosidosis | α-L-Fucosidase |
| α-Mannosidosis | α-Mannosidase |
| β-Mannosidosis | β-Mannosidase |
| Aspartylglucosaminuria | Aspartylglucosaminidase |
| M. Schindler | α-N-Acetylgalactosaminidase |
| Sialidosis (Mucolipidosis Type I) | α-Neuraminidase (Sialidase) |
| Galactosialidosis | Cathepsin A |
| GM₁-Gangliosidosis/MPS IVB | β-Galactosidase |
| GM₂-Gangliosidosis | |
| M. Sandhoff | β-Hexosaminidase A + B |
| M. Tay-Sachs | β-Hexosaminidase A |
| Metachromatic Leukodystrophy | Arylsulfatase A |
| M. Krabbe | Cerebrosid-β-Galactosidase |
| M. Gaucher | β-Glucocerebrosidase |
| M. Fabry | α-Galactosidase A |
| | (Ceramidtrihexosidase) |
| M. Niemann-Pick Type = A + B | Sphingomyelinase |
| Glycogen storage disease type II | Acid α-Glucosidase |
| (M. Pompe) | (Acid Maltase) |
| Infantile NCL (CLN1-defect) | Palmitoyl-Protein Thioesterase 1 |
| | (PPT1) |
| Late Infantile NCL (CLN2-defect) | Tripeptidyl-Peptidase 1 (TPP1) |
| Late Infantile NCL (CLN5-defect) | CLN5-Protein |
| Cathepsin D deficient NCL (CLN10- | Cathepsin D |
| defect) | |
| M. Faber | Ceramidase |
| M. Wolman | acid Lipase |
| X-chromosomal lchthyosis | Steroidsulfatase (Arylsulfatase C) |

In a preferred embodiment the chimeric molecule for use in the treatment of a disease is administered intraventricularly, preferably by use of an Ommaya reservoir or a Rickham capsule.

In one embodiment the invention relates to the use of the chimeric molecule according to the invention for the manufacture of a medicament.

The invention also relates to a method of treating a lysosomal storage disease comprising the administration of a therapeutically effective amount of a chimeric molecule according to the invention. In a preferred embodiment of the present invention the lysosomal storage disease is a lysosomal storage disease with brain involvement

In a first aspect the present invention relates to a chimeric molecule comprising
(i) a targeting moiety that binds to heparin or heparan sulfate proteoglycans,
(ii) a lysosomal peptide or protein,
(iii) wherein the targeting moiety is a neurotrophic growth factor and/or, wherein the targeting moiety comprises one of the following consensus sequences BBXB, BXBB, BBXXB, BXXBB, BBXXXB or BXXXBB and wherein B represents an arginine, lysine or histidine amino acid and X represents any amino acid,
(iv) with the proviso that the targeting moiety is at least thirteen amino acids long.

In a second aspect the present invention relates to a chimeric molecule according to the first aspect, wherein the targeting moiety is selected from the group of
(v) annexin II comprising the amino acid sequence according to SEQ ID NO. 1 (KIRSEFKKKYGKSLYY),
(vi) vitronectin comprising the amino acid sequence according to SEQ ID NO. 2 (QRFRHRNRKGYRSQRG),
(vii) ApoB comprising the amino acid sequence according to SEQ ID NO. 3 (KFIIPSPKRPVKLLSG),
(viii) bFGF comprising the amino acid sequence according to SEQ ID NO. 4 (GHFKDPKRLYCKNGGF),
(ix) NCAM comprising the amino acid sequence according to SEQ ID NO. 5 (DGGSPIRHYLIKYKAK),
(x) Protein C inhibitor comprising the amino acid sequence according to SEQ ID NO. 6 (GLSEKTLRKWLKMFKK),
(xi) AT-III comprising the amino acid sequence according to SEQ ID NO. 7 (KLNCRLYRKANKSSKL),
(xii) ApoE comprising the amino acid sequence according to SEQ ID NO. 8 (SHLRKLRKRLLRDADD),
(xiii) Fibrin comprising the amino acid sequence according to SEQ ID NO. 9 (GHRPLDKKREEAPSLR),
(xiv) hGDNF comprising the amino acid sequence according to SEQ ID NO. 10 (SRGKGRRGQRGKNRG),
(xv) B-thromboglobulin comprising the amino acid sequence according to SEQ ID NO. 11 (PDAPRIKKIVQKKLAG)
(xvi) Insulin-like growth factor-binding protein-3 comprising the amino acid sequence according to SEQ ID NO. 12 (DKKGFYKKKQCRPSKG),
(xvii) Antp comprising the amino acid sequence according to SEQ ID NO. 13 (RQIKIWFQNRRMKWKK)
(xviii) human clock comprising the amino acid sequence according to SEQ ID NO. 14 (KRVSRNKSEKKRR)

In a third aspect the present invention relates to a chimeric molecule according to the first or the second aspect, wherein the growth factor is modified and lysosomal targeting is improved.

In a fourth aspect the present invention relates to a chimeric molecule according to any one of the aspects from the first to the third aspect, wherein the targeting moiety and the enzyme moiety are covalently linked to each other.

In a fifth aspect the present invention relates to a chimeric molecule according to any one of the aspects from the first to the fourth aspect, wherein the chimeric molecule is a single polypeptide chain.

In a sixth aspect the present invention relates to a chimeric molecule according to any one of the aspects from the first to the fifth aspect, wherein the targeting moiety and the enzyme moiety are linked via a peptide linker.

In a seventh aspect the present invention relates to the chimeric molecule according to any one of the aspects from the first to the sixth aspect, wherein the peptide linker comprises a protease cleavage site.

In an eighth aspect the present invention relates to a chimeric molecule according to any one of the aspects from the first to the seventh aspect, wherein the protease cleavage site is that of a protease selected from the group consisting of factor Xa, thrombin, trypsin, papain and plasmin.

In a ninth aspect the present invention relates to a chimeric molecule according to any one of the aspects from the first to the eighth aspect, wherein the lysosomal enzyme is selected from the group consisting of, β-galactocerebrosidase, arylsulfatase A (sulfatidase), α-iduronidase, sulfaminidase, α-N-acetylglucosaminidase, acetyl-CoA:α-glucosaminide-N-Ac-transferase, N-acetylglucosamine-6-sulfatase, tripeptidyl-peptidase 1, palmitoyl-protein thioesterase, β-galactosidase, sphingomyelinase, β-hexosaminidase A, β-hexosaminidase A+B, ceramidase, α-mannosidase, β-mannosidase, α-fucosidase, sialidase, α-N-acetylgalactosaminidase, α-L-iduronidase, iduronate-2-sulfatase, sulfamidase (heparan N-sulftase), α-N-acetylglucosaminidase, N-acetylgalactosamin-6-sulfatase, arylsulfatase B, β-glucuronidase, α-L-fucosidase, aspartylgiucosaminidase, α-neuraminidase (sialidase), cathepsin A, arylsulfatase A, cerebroside-β-galactosidase, β-glucocerebrosidase, α-galactosidase A (ceramide trihexosidase), acid α-glucosidase (acid maltase), CLN5-protein, acid lipase, steroid sulfatase (arylsulfatase C) and cathepsin D.

In a tenth aspect the present invention relates to a chimeric molecule according to any one of aspects from the second to the ninth aspect, wherein the targeting moiety is a polypeptide having a sequence according to any one of SEQ ID NO. 18, 20, 24, 26, 28 and 30.

In a eleventh aspect the present invention relates to a molecule according to any one of the aspects from the first to the tenth aspect, wherein the enzyme moiety (lysosomal protein or peptide) is a polypeptide having a sequence according to any one of SEQ ID NO. 52, 54, 56, 58, 60, 62, 64, 66, 68 and 70.

In a twelfth aspect the present invention relates to a chimeric molecule according to the tenth or the eleventh aspect, wherein the polypeptide has a sequence according to any one of the SEQ ID NO. 32, 34, 36, 38, 40, 42, 46, 48 and 50.

In a thirteenth aspect the present invention relates to a polynucleotide encoding the chimeric molecule according to any one of the aspects from the first to the twelfth aspect.

In a fourteenth aspect the invention relates to a polynucleotide according to thirteenth aspect having the sequence according to any one of the SEQ ID NO. 31, 33, 35, 37, 39, 41, 45, 47 and 49.

In a fifteenth aspect the present invention relates to a pharmaceutical composition comprising a chimeric molecule according to any one of the aspect from the first to the twelfth aspect.

In a sixteenth aspect the present invention relates to a chimeric molecule according to any one of the aspects from the first to the twelfth aspect for the use in the treatment of a disease.

In a seventeenth aspect the present invention relates to a chimeric molecule according for the use in the treatment of a disease according to the sixteenth aspect, wherein the disease is a lysosomal storage disease.

In an eighteenth aspect the present invention relates to a chimeric molecule for the use in the treatment of a disease according to the seventeenth aspect, wherein the lysosomal storage disease is selected from the group consisting of the neuronal ceroid lipofuscinoses (NCL), infantile NCL (CLN1-defect), late infantile NCL (CLN2-defect), late infantile NCL (CLN5-defect), NCL caused by cathepsin D deficiency (CLN10-defect), mucopolysaccharidosis type I, mucopolysaccharidosis type II, mucopolysaccharidosis type IIIA, mucopolysaccharidosis type IIIB, mucopolysaccharidosis type IIIC, mucopolysaccharidosis type IIID, mucopolysaccharidosis type IVA, mucopolysaccharidosis type IVB, mucopolysaccharidosis type VI, mucopolysaccharidosis type VII, fucosidosis, mannosidosis, β-mannosidosis, aspartylglucosaminuria, Schindler's disease, Sialidosis (Mucolipidosis I), galaktosialidosis, GM₁-gangliosidosis/mucopolysaccharidosis type IVB, GM₂-gangliosidosis, Sandhoff disease, Tay-Sachs disease, metachromatic leukodystrophy, Krabbe disease, Gaucher disease, Fabry disease, Niemann-Pick disease typeA+B, glycogen storage disease type II (Pompe disease), Faber's syndrome, Wolman disease, X-linked ichthyosis.

In a nineteenth aspect present invention relates to a chimeric molecule for the use in the treatment of a disease according to the eighteenth aspect, wherein the lysosomal storage disease is the late infantile form of neuronal ceroid lipofuscinosis and the enzyme moiety comprises lysosomal tripeptidyl peptidase 1 (TPP1).

In a twentieth aspect the present invention relates to a chimeric molecule for the use in the treatment of a disease according to any one of the aspects from the sixteenth to the nineteenth aspect, wherein the chimeric molecule is administered intraventricularly, by use of an Ommaya reservoir, a Rickham capsule or a similar device known by those skilled in the art.

In a twenty-first aspect the present invention relates to the use of the chimeric molecule according to any one of the aspects from the first to the twelfth aspect for the manufacture of a medicament.

In a twenty-second aspect the present invention relates to a method of treating a lysosomal storage disease comprising the administration of a therapeutically effective amount of a chimeric molecule according to any one of the aspects from the first to the twelfths aspect to a subject.

### EXAMPLES

### Example 1

The medium to be purified is adjusted to a pH-value of 6.0 using a phosphate buffer (final concentration 20 mM; stock solution: KH₂PO₄, 1 M, pH 4.5 and K₂HPO₄ 1 M pH 9). After centrifugation for 10 min at 40.000 g and 4° C., the medium is filtrated through a 0.2 μm filter and then degassed. The supernatant, having a maximum NaCl concentration of 100 mM, is applied to a cation exchange column (for example Resource S). The flow-through is collected.

The column is then washed with 10 column volumes of a 20 mM phosphate buffer (pH 6, 100 mM NaCl). A further washing step using an intermediate gradient of 100 to 150 mM NaCl over 5 column volumes is applied. Elution is achieved by applying a linear gradient of 150 to 500 mM NaCl over 20 column volumes (1 ml fractions are collected). A final step of 1 M NaCl over 10 column volumes is applied. UV and salt gradient are monitored during the entire elution process.

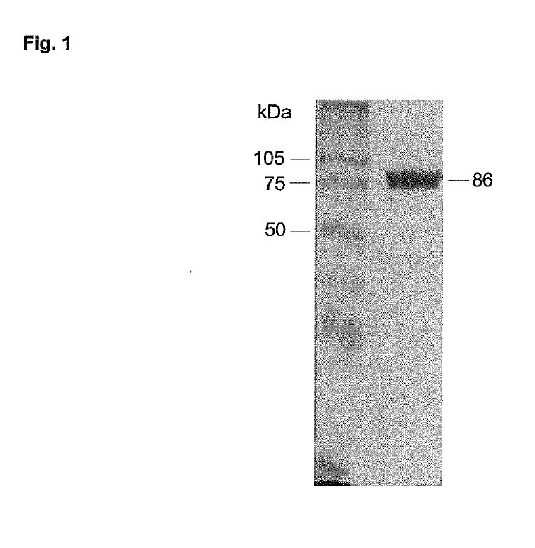

Fractions containing the fusion protein are pooled and adjusted to pH 7.5 using phosphate buffer. FIG. 1 shows a purified sample of the TPP1-FGF2 fusion protein.

### Example 2

The medium is adjusted to a pH of 7.5 using a 20 mM phosphate buffer, centrifuged for 10 min at 40.000 g and 4° C., filtrated through a 0.2 μm filter and then degassed. The supernatant is diluted with 1 volume of 20 mM phosphate buffer (pH 7.5) so that the diluted supernatant has a maximum NaCl concentration of 80 mM. The diluted supernatant is then applied to an anion exchange column (for example Resource Q). The column is subsequently washed with 10 column volumes of phosphate buffer (pH 7.5; 80 mM NaCl) followed by an intermediate NaCl gradient of 80 to 150 mM NaCl over 10 column volumes. For elution, the a linear gradient of 150-500 mM NaCl over 20 column volumes is applied (1 ml fractions are collected, peak between 200-300 mM NaCl) with a subsequent adjustment to 1 M NaCl over 10 column volumes. UV and salt gradient are monitored during the entire elution process.

### Example 3

The medium is adjusted to a pH of 7.5 using 20 mM phosphate buffer. The final NaCl concentration is adjusted to 800 mM NaCl. The medium is then centrifuged for 10 min at 40.000 g and 4° C., followed by filtration through a 0.2 μm filter and subsequent degassing. The filtered supernatant is then applied to a Heparin-Sepharose-column (flow rate 1 ml/min), the flow-through is collected.

Purification is continued by applying 10 column volumes of 20 mM phosphate buffer (pH 7.5, 800 mM NaCl). For elution a linear gradient of 0.8-2 M NaCl over 20 column volumes is applied (1 ml fractions are collected, peak between 1.5 and 1.8 M NaCl), followed by a 2 M NaCl step over 10 column volumes. UV and salt gradient are monitored during the entire elution process.

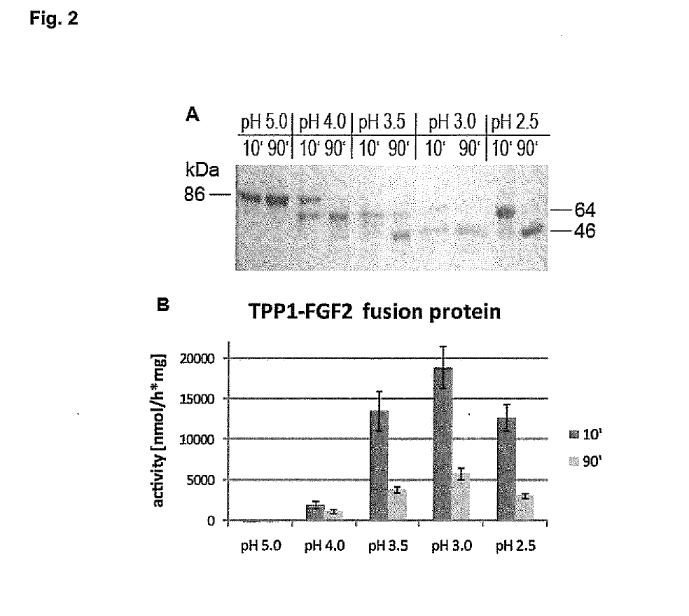

After subsequent desalting and buffer exchange to PBS (pH 7.5) using gel filtration or ultrafiltration, the TPP1-FGF2 fusion proteins are aliquoted and stored at −70° C. For further characterisation of the fusion proteins, the enzymatic activities are examined by a standardized enzyme assay. The pH dependent auto-activation of the TPP1-FGF2 proproteins was comparable to that of the TPP1 wild-type. FIG. 2 illustrates the auto-processing of a TPP1-FGF2 fusion protein.

### Example 4

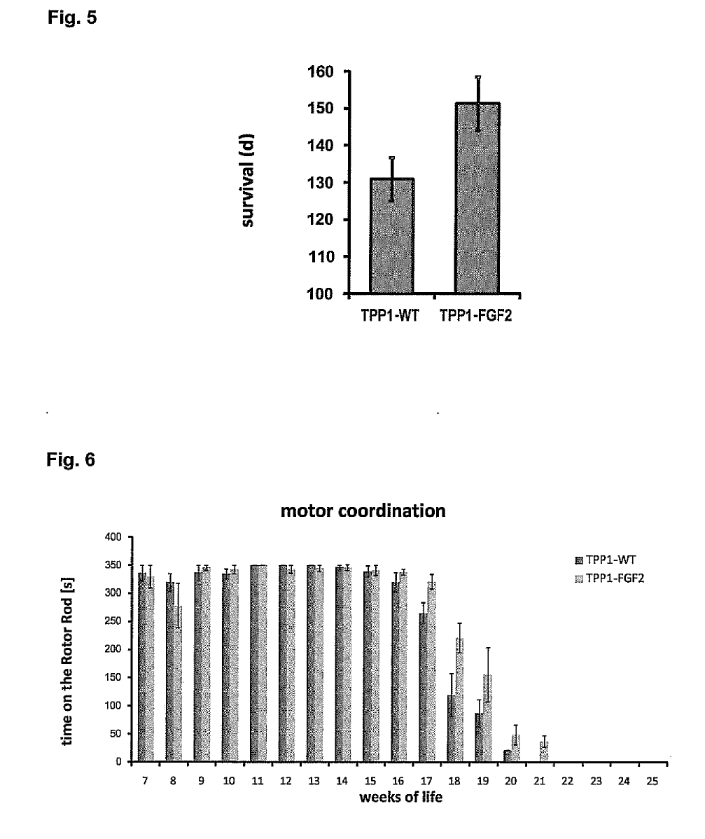

Furthermore, endocytosis into human neuronal progenitor cells (NT2 cells) was compared for TPP1-FGF2 fusion proteins and the TPP1 wild-type. At a final concentration of 0.4-0.5 μM TPP1-FGF2 fusion protein or TPP1 wild-type protein, respectively, was added to the medium. After 48 hours of incubation the intracellular TPP1-activity was measured (see FIG. 4). TPP1-activity was six times higher in cell lysates of the NT2-cells which were treated with TPP1-FGF2 fusion proteins than for the TPP1 wild-type protein. It was possible to inhibit the intracellular TPP1-activity by heparin either alone or in combination with mannose-6-phosphate. The results show that the cellular uptake of the TPP1-FGF2 fusion protein is mainly mediated by cell surface HSPG.

### Example 5

Finally, the effect of the TPP1-FGF1 fusion proteins was also examined in an animal model, namely tpp1−/− mice. In weekly intervals, the tpp1−/− mice were injected intraventricularly with 10 μg of TPP1-FGF2 fusion protein or TPP1 wild-type protein, respectively. Mice treated with TPP1-FGF2 showed a significantly higher life expectancy as compared to mice treated with the TPP1 wild-type protein (FIG. 5).

Moreover, tpp1−/− mice treated with TPP1-FGF2-fusion proteins showed a delayed course of illness in comparison to tpp1−/− mice treated with the TPP1 wild-type. This result was tested by checking the motor coordination with a so called Rotor Rod (a rotating pole) (FIG. 6). As of the 17^{th} week, tpp1−/− mice treated with the TPP1-FGF2 fusion protein were able to stay longer on the Rotor Rod than the tpp1−/− mice treated with the TPP1 wild-type.

1. A chimeric molecule comprising
(i) a targeting moiety that binds to heparin or heparan sulfate proteoglycans, and
(ii) a lysosomal peptide or protein, wherein the lysosomal peptide or protein is tripeptidyl-peptidase 1,
wherein the targeting moiety is Basic Fibroblast Growth Factor (bFGF) comprising the amino acid sequence according to SEQ ID NO. 4 (GHFKDPKRLYCKNGGF).

2. Chimeric molecule according to claim 1, wherein the growth factor is modified and lysosomal targeting is improved.

3. Chimeric molecule according to claim 1, wherein the targeting moiety and the enzyme moiety are covalently linked to each other.

4. Chimeric molecule according to claim 1, wherein the chimeric molecule is a single polypeptide chain.

5. Chimeric molecule according to claim 1, wherein the targeting moiety and the enzyme moiety are linked via a peptide linker.

6. Chimeric molecule according to claim 1, wherein the peptide linker comprises a protease cleavage site.

7. Chimeric molecule according to claim 1, wherein the protease cleavage site is that of a protease selected from the group consisting of factor Xa, thrombin, trypsin, papain and plasmin.

8. Chimeric molecule according to claim 1, wherein the targeting moiety is a polypeptide having a sequence according to any one of SEQ ID NO. 24, 26, 28 and 30.

9. Chimeric molecule according to claim 1, wherein the enzyme moiety is a polypeptide having a sequence according to SEQ ID NO. 52.

10. Chimeric molecule according to claim 8 or 9, wherein the polypeptide has a sequence according to any one of the SEQ ID NO. 36, 38, 40, and 42.

11. Polynucleotide encoding the chimeric molecule according to claim 1.

12. Polynucleotide according to claim 11 having the sequence according to any one of the SEQ ID NO. 35, 37, 39, and 41.

13. Pharmaceutical composition comprising a chimeric molecule according to claim 1.

14. (canceled)

15. Method of treating a lysosomal storage disease comprising administering a pharmaceutically effective amount of the pharmaceutical composition of claim 13 to a patient in need thereof.

16. The method according to claim 14, wherein the lysosomal storage disease is selected from the group consisting of the neuronal ceroid lipofuscinoses (NCL), infantile NCL (CLN1-defect), late infantile NCL (CLN2-defect), late infantile NCL (CLN5-defect), NCL caused by cathepsin D deficiency (CLN10-defect).

17. The method according to claim 14 or 15, wherein the chimeric molecule is administered intraventricularly, by use of an Ommaya reservoir, a Rickham capsule or a similar device.

18. (canceled)


SEQUENCE LISTING (74 sequences)

SEQ ID NO: 1 - protein, 16 aa, Homo sapiens
KIRSEFKKKYGKSLYY

SEQ ID NO: 2 - protein, 16 aa, Homo sapiens
QRFRHRNRKGYRSQRG

SEQ ID NO: 3 - protein, 16 aa, Homo sapiens
KFIIPSPKRPVKLLSG

SEQ ID NO: 4 - protein, 16 aa, Homo sapiens
GHFKDPKRLYCKNGGF

SEQ ID NO: 5 - protein, 16 aa, Homo sapiens
DGGSPIRHYLIKYKAK

SEQ ID NO: 6 - protein, 16 aa, Homo sapiens
GLSEKTLRKWLKMFKK

SEQ ID NO: 7 - protein, 16 aa, Homo sapiens
KLNCRLYRKANKSSKL

SEQ ID NO: 8 - protein, 16 aa, Homo sapiens
SHLRKLRKRLLRDADD

SEQ ID NO: 9 - protein, 16 aa, Homo sapiens
GHRPLDKKREEAPSLR

SEQ ID NO: 10 - protein, 15 aa, Homo sapiens
SRGKGRRGQRGKNRG

SEQ ID NO: 11 - protein, 16 aa, Homo sapiens
PDAPRIKKIVQKKLAG

SEQ ID NO: 12 - protein, 16 aa, Homo sapiens
DKKGFYKKKQCRPSKG

SEQ ID NO: 13 - protein, 16 aa, Homo sapiens
RQIKIWFQNRRMKWKK

SEQ ID NO: 14 - protein, 13 aa, Homo sapiens
KRVSRNKSEKKRR

SEQ ID NO: 15 - DNA, 12 bp, Homo sapiens
agatcccccggg

SEQ ID NO: 16 - DNA, 12 bp, Homo sapiens
ggatcccccggg

SEQ ID NO: 17 - DNA, 51 bp, Homo sapiens
cgccagataaagatttggttccagaatcggcgcatgaagtggaagaagtaa

SEQ ID NO: 18 - protein, 16 aa, Homo sapiens
RQIKIWFQNRRMKWKK

SEQ ID NO: 19 - DNA, 42 bp, Homo sapiens
aaaagagtatctagaaacaaatctgaaaagaaacgtagataa

SEQ ID NO: 20 - protein, 13 aa, Homo sapiens
KRVSRNKSEKKRR

SEQ ID NO: 21 - DNA, 30 bp, Homo sapiens
agatccgtcgacatcgaaggtagaggcatt

SEQ ID NO: 22 - DNA, 30 bp, Homo sapiens
ggatccgtcgacatcgaaggtagaggcatt

SEQ ID NO: 23 - DNA, 441 bp, Homo sapiens
cccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaatctgttacggatgagtgtttcttttttgaacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 24 - protein, 145 aa, Homo sapiens
PALPEDGGSGAFPPGHFKDPKRLYCKNGGFFLRIHPDGRVDGVREKSDPHIKLQLQAEERGVVSIKGVSANRYLAMKEDGRLLASKSVTDECFFFERLESNNYNTYRSRKYTSWYVALKRTGQYKLGSKTGPGQKAILFLPMSAK

SEQ ID NO: 25 - DNA, 441 bp, Homo sapiens
cccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaatctgttacggatgagtgtttcttttttgcacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 26 - protein, 146 aa, Homo sapiens
PALPEDGGSGAFPPGHFKDPKRLYCKNGGFFLRIHPDGRVDGVREKSDPHIKLQLQAEERGVVSIKGVSANRYLAMKEDGRLLASKSVTDECFFFARLESNNYNTYRSRKYTSWYVALKRTGQYKLGSKTGPGQKAILFLPMSAKS

SEQ ID NO: 27 - DNA, 441 bp, Homo sapiens
cccgccttgcccgaggatggcggcagcggcgccttcccgcccggccacttcaaggaccccaagcggctgtactgcaaaaacgggggcttcttcctgcgcatccaccccgacggccgagttgacgggacaagggacaggagcgaccagcacattcagctgcagctcagtgcagaagagagaggagttgtgtctatcaaaggagtgtctgctaaccgttacctggctatgaaggaagatggaagattactggcttctaaatctgttacggatgagtgtttcttttttgaacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 28 - protein, 146 aa, Homo sapiens
PALPEDGGSGAFPPGHFKDPKRLYCKNGGFFLRIHPDGRVDGTRDRSDQHIQLQLSAEERGVVSIKGVSANRYLAMKEDGRLLASKSVTDECFFFERLESNNYNTYRSRKYTSWYVALKRTGQYKLGSKTGPGQKAILFLPMSAKS

SEQ ID NO: 29 - DNA, 441 bp, Homo sapiens
cccgccttgcccgaggatggcggcagcggcgccttcccgcccggccacttcaaggaccccaagcggctgtactgcaaaaacgggggcttcttcctgcgcatccaccccgacggccgagttgacgggacaagggacaggagcgaccagcacattcagctgcagctcagtgcagaagagagaggagttgtgtctatcaaaggagtgtctgctaaccgttacctggctatgaaggaagatggaagattactggcttctaaatctgttacggatgagtgtttcttttttgcacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 30 - protein, 146 aa, Homo sapiens
PALPEDGGSGAFPPGHFKDPKRLYCKNGGFFLRIHPDGRVDGTRDRSDQHIQLQLSAEERGVVSIKGVSANRYLAMKEDGRLLASKSVTDECFFFARLESNNYNTYRSRKYTSWYVALKRTGQYKLGSKTGPGQKAILFLPMSAKS

SEQ ID NO: 31 - DNA, 1752 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatcccccgggcgccagataaagatttggttccagaatcggcgcatgaagtggaagaagtaa

SEQ ID NO: 32 - protein, 583 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPRSPGRQIKIWFQNRRMKWKK

SEQ ID NO: 33 - DNA, 1743 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatcccccgggaaaagagtatctagaaacaaatctgaaaagaaacgtagataa

SEQ ID NO: 34 - protein, 580 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPRSPGKRVSRNKSEKKRR

SEQ ID NO: 35 - DNA, 2160 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatccgtcgacatcgaaggtagaggcattcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaatctgttacggatgagtgtttcttttttgaacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 36 - protein, 719 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPRSVDIEGRGIPALPEDGGSGAFPPGHFKDPKRLYCKNGGFFLRIHPDGRVDGVREKSDPHIKLQLQAEERGVVSIKGVSANRYLAMKEDGRLLASKSVTDECFFFERLESNNYNTYRSRKYTSWYVALKRTGQYKLGSKTGPGQKAILFLPMSAKS

SEQ ID NO: 37 - DNA, 2160 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatccgtcgacatcgaaggtagaggcattcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaatctgttacggatgagtgtttcttttttgcacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 38 - protein, 719 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPRSVDIEGRGIPALPEDGGSGAFPPGHFKDPKRLYCKNGGFFLRIHPDGRVDGVREKSDPHIKLQLQAEERGVVSIKGVSANRYLAMKEDGRLLASKSVTDECFFFARLESNNYNTYRSRKYTSWYVALKRTGQYKLGSKTGPGQKAILFLPMSAKS

SEQ ID NO: 39 - DNA, 2160 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatccgtcgacatcgaaggtagaggcattcccgccttgcccgaggatggcggcagcggcgccttcccgcccggccacttcaaggaccccaagcggctgtactgcaaaaacgggggcttcttcctgcgcatccaccccgacggccgagttgacgggacaagggacaggagcgaccagcacattcagctgcagctcagtgcagaagagagaggagttgtgtctatcaaaggagtgtctgctaaccgttacctggctatgaaggaagatggaagattactggcttctaaatctgttacggatgagtgtttcttttttgaacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 40 - protein, 719 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPRSVDIEGRGIPALPEDGGSGAFPPGHFKDPKRLYCKNGGFFLRIHPDGRVDGTRDRSDQHIQLQLSAEERGVVSIKGVSANRYLAMKEDGRLLASKSVTDECFFFERLESNNYNTYRSRKYTSWYVALKRTGQYKLGSKTGPGQKAILFLPMSAKS

SEQ ID NO: 41 - DNA, 2160 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaaccccagatccgtcgacatcgaaggtagaggcattcccgccttgcccgaggatggcggcagcggcgccttcccgcccggccacttcaaggaccccaagcggctgtactgcaaaaacgggggcttcttcctgcgcatccaccccgacggccgagttgacgggacaagggacaggagcgaccagcacattcagctgcagctcagtgcagaagagagaggagttgtgtctatcaaaggagtgtctgctaaccgttacctggctatgaaggaagatggaagattactggcttctaaatctgttacggatgagtgtttcttttttgcacgattggaatctaataactacaatacttaccggtcaaggaaatacaccagttggtatgtggcactgaaacgaactgggcagtataaacttggctccaaaacaggacctgggcagaaagctatactttttcttccaatgtctgctaagagctga

SEQ ID NO: 42 - protein, 719 aa, Homo sapiens [sequence omitted]

SEQ ID NO: 43 - DNA, 276 bp, Homo sapiens
atgcagccctccagccttctgccgctcgccctctgcctgctggctgcacccgccggatcttccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcggatct

SEQ ID NO: 44 - DNA, 141 bp, Homo sapiens
atgcagccctccagccttctgccgctcgccctctgcctgctggctgcacccgccggatctgggaaaagaaagaagaaaggcaaggggctagggaagaagagggacccatctcttcggaaatacaaggacttctccggatct

SEQ ID NO: 45 - DNA, 1725 bp, Homo sapiens
atgcagccctccagccttctgccgctcgccctctgcctgctggctgcacccgccggatcttccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcggatctcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtaggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcactacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtaa

SEQ ID NO: 46 - protein, 574 aa, Homo sapiens
MQPSSLLPLALCLLAAPAGSSKPQALATPNKEEHGKRKKKGKGLGKKRDPCLRKYKDFCIHGECKYVKELRAPSCICHPGYHGERCHGLSGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 47 - DNA, 1590 bp, Homo sapiens
atgcagccctccagccttctgccgctcgccctctgcctgctggctgcacccgccggatctgggaaaagaaagaagaaaggcaaggggctagggaagaagagggacccatctcttcggaaatacaaggacttctccggatctcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtaggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcactacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtaa

SEQ ID NO: 48 - protein, 529 aa, Homo sapiens
MQPSSLLPLALCLLAAPAGSGKRKKKGKGLGKKRDPSLRKYKDFSGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 49 - DNA, 1650 bp, Homo sapiens
atgcagccctccagccttctgccgctcgccctctgcctgctggctgcacccgccggatctgggaaaagaaagaagaaaggcaaggggctagggaagaagagggacccatctcttcggaaatacaaggacttctccggatctcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtaggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcactacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgagatcccccgggcgccagataaagatttggttccagaatcggcgcatgaagtggaagaagtaa

SEQ ID NO: 50 - protein, 549 aa, Homo sapiens
MQPSSLLPLALCLLAAPAGSGKRKKKGKGLGKKRDPSLRKYKDFSGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRSPGRQIKIWFQNRRMKWKK

SEQ ID NO: 51 - DNA, 1692 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

SEQ ID NO: 52 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 53 - DNA, 1239 bp, Homo sapiens
atgcagccctccagccttctgccgctcgccctctgcctgctggctgcacccgcctccgcgctcgtcaggatcccgctgcacaagttcacgtccatccgccggaccatgtcggaggttgggggctctgtggaggacctgattgccaaaggccccgtctcaaagtactcccaggcggtgccagccgtgaccgaggggcccattcccgaggtgctcaagaactacatggacgcccagtactacggggagattggcatcgggacgcccccccagtgcttcacagtcgtcttcgacacgggctcctccaacctgtgggtcccctccatccactgcaaactgctggacatcgcttgctggatccaccacaagtacaacagcgacaagtccagcacctacgtgaagaatggtacctcgtttgacatccactatggctcgggcagcctctccgggtacctgagccaggacactgtgtcggtgccctgccagtcagcgtcgtcagcctctgccctgggcggtgtcaaagtggagaggcaggtctttggggaggccaccaagcagccaggcatcaccttcatcgcagccaagttcgatggcatcctgggcatggcctacccccgcatctccgtcaacaacgtgctgcccgtcttcgacaacctgatgcagcagaagctggtggaccagaacatcttctccttctacctgagcagggacccagatgcgcagcctgggggtgagctgatgctgggtggcacagactccaagtattacaagggttctctgtcctacctgaatgtcacccgcaaggcctactggcaggtccacctggaccaggtggaggtggccagcgggctgaccctgtgcaaggagggctgtgaggccattgtggacacaggcacttccctcatggtgggcccggtggatgaggtgcgcgagctgcagaaggccatcggggccgtgccgctgattcagggcgagtacatgatcccctgtgagaaggtgtccaccctgcccgcgatcacactgaagctgggaggcaaaggctacaagctgtccccagaggactacacgctcaaggtgtcgcaggccgggaagaccctctgcctgagcggcttcatgggcatggacatcccgccacccagcgggccactctggatcctgggcgacgtcttcatcggccgctactacactgtgtttgaccgtgacaacaacagggtgggcttcgccgaggctgcccgcctctag

SEQ ID NO: 54 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 55 - DNA, 921 bp, Homo sapiens
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 56 - protein, 306 aa, Homo sapiens
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 57 - DNA, 1509 bp, Homo sapiens
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtaggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcactacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 58 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 59 - DNA, 1962 bp, Homo sapiens
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 60 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 61 - DNA, 1653 bp, Homo sapiens
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 62 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 63 - DNA, 1524 bp, Homo sapiens
atgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctga

SEQ ID NO: 64 - protein, 507 aa, Homo sapiens
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 65 - DNA, 2010 bp, Homo sapiens
atggctgcagccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagatggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacgtgggctgccgatgcatccaacacaatcagtattataggagactacaactggaccaatctgactacaaagtgtgatgtttacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcgcctctggcatgctgaatgacaagtctctgtggacagacatccctgtgaattttccaaagaatggctgggctgcaattggaactcactcctttgaatttgcacagtttgacaactttcttgtggaagccacacgctaa

SEQ ID NO: 66 - protein, 669 aa, Homo sapiens
MAAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTTKCDVYIETPDTGGVFIAGRVNKGGILIRSARGIFFWIFANGSYRVTGDLAGWIIYALGRVEVTAKKWYTLTLTIKGHFASGMLNDKSLWTDIPVNFPKNGWAAIGTHSFEFAQFDNFLVEATR

SEQ ID NO: 67 - DNA, 1611 bp, Homo sapiens
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagcagcgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagagcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggccagcgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 68 - protein, 538 aa, Homo sapiens
ASMEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 69 - DNA, 1320 bp, Homo sapiens
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttgggaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaatgcagatgtcattaaaagacttactttaa

SEQ ID NO: 70 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 71 - protein, 92 aa, Homo sapiens
MQPSSLLPLALCLLAAPAGSSKPQALATPNKEEHGKRKKKGKGLGKKRDPCLRKYKDFCIHGECKYVKELRAPSCICHPGYHGERCHGLSGS

SEQ ID NO: 72 - protein, 47 aa, Homo sapiens
MQPSSLLPLALCLLAAPAGSGKRKKKGKGLGKKRDPSLRKYKDFSGS

SEQ ID NO: 73 - DNA, 30 bp, Artificial Sequence
  N-terminal Linker
agatccgtcgacatcgaaggtagcggcatt

SEQ ID NO: 74 - DNA, 30 bp, Artificial Sequence
  N-terminal Linker
ggatccgtcgacatcgaaggtagcggcatt